(19) United States
(12) Patent Application Publication
(10) Pub. No.: US 2023/0310452 A1
(43) Pub. Date: Oct. 5, 2023

(54) BROMODOMAIN AND EXTRA TERMINAL DOMAIN (BET) INHIBITOR COMPOSITIONS AND METHODS THEREOF FOR USE AS ANTI-AGING AGENTS

(71) Applicant: MDI Biological Laboratory, Bar Harbor, ME (US)

(72) Inventors: Samyeol BAEK, Bar Harbor, ME (US); Jarod ROLLINS, Bar Harbor, ME (US)

(73) Assignee: MDI Biological Laboratory, Bar Harbor, ME (US)

(21) Appl. No.: 18/022,948

(22) PCT Filed: Aug. 24, 2021
(86) PCT No.: PCT/US2021/047229

§ 371 Date: Feb. 23, 2023

Related U.S. Application Data
(60) Provisional application No. 63/070,426, filed on Aug. 26, 2020.

(51) Int. Cl. A61P 29/00; A61K 31/5517

(52) U.S. Cl. CPC A61K 31/5517; A61P 29/00

(57) ABSTRACT

Pharmaceutical compositions comprising a bromodomain and extraterminal domain (BET) inhibitor, methods, and systems for screening candidate substances or compounds thereof, for the prevention and/or treatment of age-related diseases, conditions, or disorders to a subject in need thereof. The invention also provides pharmaceutical compositions for treating or preventing an aging or age-related disease, condition, or disorder comprising a BET inhibitor and a pharmaceutically acceptable carrier.

### RELATED APPLICATION

This application claims the benefit of U.S. Provisional Pat. Application No. 63/070,426, filed Aug. 26, 2020, to “BROMODOMAIN AND EXTRA TERMINAL DOMAIN (BET) INHIBITOR COMPOSITIONS AND METHODS THEREOF FOR USE AS ANTI-AGING AGENTS,” which is incorporated herein by reference in its entirety for all purposes.

### STATEMENT OF RIGHTS TO INVENTIONS MADE UNDER FEDERALLY SPONSORED RESEARCH

This invention was made with government support under Grant No. 2P20GM104318-06 awarded by the National Institute of General Medical Sciences (NIGMS), National Institutes of Health (NIH). The government has certain rights in the invention.

### BACKGROUND OF THE INVENTION

Aging is a progressive process that occurs in cells, organs, and whole organisms. The global anti-aging market reached US$ 50.2 billion in 2018 and is further projected to reach a value of US$ 79.5 billion by 2024, growing at a compound annual growth rate (CAGR) of 7.98% during 2019-2024. Accordingly, there is a great desire to extend one’s life and lifespan, particularly for those looking for the proverbial “fountain of youth.” Individuals attempt to stave off aging and increase life expectancy by improving health care and nutrition. However, these routes demand consistency, time, and effort; whereas taking a pill would be a simple and convenient method.

Therefore, there is a great desire and need for effective candidate substances and methods of treating aging or age-related diseases or disorders. There is a need for more effective treatments of age-related diseases and the need for a greater understanding of agents that may increase lifespan and delay the appearance of age-related diseases.

### SUMMARY OF THE INVENTION

As described below, the present invention features novel candidate substances, pharmaceutical compositions containing such substances, and their use in the prevention and treatment of diseases and conditions associated with aging or age-related diseases or disorders, including age-related inflammatory diseases or disorders.

One aspect provides a method of treating a subject suffering from an age-related inflammatory disease in a subject by administering a therapeutically effective amount of a Bromodomain and ExtraTerminal (BET) domain inhibitor to the subject in need thereof. The BET inhibitor for use in various aspects of the invention may be selected from, but not limited to, tert-butyl (S)-2-(4-(4-chlorophenyl)-2,3,9-trimethyl-6H-thieno[3,2-f][1,2,4]triazolo[4,3-a][1,4]diazepin-6-yl)acetate (JQ1); (R)-7-(3,5-dimethylisoxazol-4-yl)-8-methoxy-1-(1-(pyridin-2-yl)ethyl)-1,3-dihydro-2H-imidazo[4,5-c]quinolin-2-one (I-BET 151); (S)-2-(6-(4-chlorophenyl)-8-methoxy-1-methyl-4H-benzo[f][1,2,4]triazolo [4,3-a][1,4]diazepin-4-yl)-N-ethylacetamide (I-BET 762); (S)-2-(6-(4-chlorophenyl)-8-methoxy-1-methyl-4H-benzo[f][1,2,4]triazolo[4,3-a][1,4]diazepin-4-yl)-N-ethylacetamide (OTX-015); (S)-2-(4-(4-chlorophenyl)-2,3,9-trimethyl-6H-thieno[3,2-f][1,2,4]triazolo [4,3-a][1,4]diazepin-6-yl)acetamide (CPI-203); (S)-2-(6-(4-chlorophenyl)-1-methyl-4H-benzo[c] isoxazolo[4,5-e]azepin-4-yl)acetamide (CPI-0610); 2-(4-(2-hydroxyethoxy)-3,5-dimethylphenyl)-5,7-dimethoxyquinazolin-4(3H)-one (RVX-208); (Z)-4-(2-(2-amino-5-methyl-4-oxocyclohexa-2,5-dien-1-ylidene)hydrazineyl)-N-(pyridin-2-yl)benzenesulfonamide (MS436); 2-methoxy-N-(3-methyl-2-oxo-1,2,3,4-tetrahydroquinazolin-6-yl)benzenesulfonamide (PFI-1); N-ethyl-4-(2-(4-fluoro-2,6-dimethylphenoxy)-5-(2-hydroxypropan-2-yl)phenyl)-6-methyl-7-oxo-6,7-dihydro-1H-pyrrolo[2,3-c]pyridine-2-carboxamide (ABBV-744); 2-Morpholin-4-yl-8-phenylchromen-4-one (LY294002); (R)-4-(2-(4-(1-(3-methoxy-[1,2,4]triazolo[4,3-b]pyridazin-6-yl)piperidin-4-yl)phenoxy) ethyl)-1,3-dimethylpiperazin-2-one (AZD 5153); N,N′-(3,6,9,12,15,18,21-heptaoxatricosane-1,23-diyl)bis(2-((S)-4-(4-chlorophenyl)-2,3,9-trimethyl-6H-thieno[3,2-f][1,2,4]triazolo[4,3-a][1,4] diazepin-6-yl) acetamide) (MT-1); (S)-N,N′-(decane-1,10-diyl)bis(2-((6S)-4-(4-chlorophenyl)-2,3,9-trimethyl-6H-thieno[3,2-f] [1,2,4]triazolo[4,3-a][1,4]diazepin-6-yl)acetamide) (MS645); 2-(4-(2-(isopropyl amino)ethoxy)-3,5-dimethylphenyl)-5,7-dimethoxyquinazolin-4(3H)-one (RVX2135); (S)-7,8-dimethoxy-N,4-dimethyl-1-(4-(4-methylpiperazin-1-yl)phenyl)-4,5-dihydro-3H-benzo[d] [1,2] diazepine-3-carboxamide (BAY1238097); (S)-6-(3,5-dimethylisoxazol-4-yl)-3-(pyridin-2-yl)-3,4-dihydro-5-oxa-1,2a-diazaacenaphthylen-2(1H)-one (INCB054329); 6-(3-hydroxypropyl)-2-(1,3,6-trimethyl-2-oxo-2,3-dihydro-1H-benzo[d]imidazol-5-yl)-1H-benzo[de]isoquinoline-1,3(2H)-dione (BAY-299); (S)-2-(3-(1,4-dimethyl-1H-1,2,3-triazol-5-yl)-5-(phenyl(tetrahydro-2H-pyran-4-yl) methyl)-5H-pyrido[3,2-b]indol-7-yl)propan-2-ol (BMS-986158); N-(4-(2,4-difluorophenoxy)-3-(6-methyl-7-oxo-6,7-dihydro-1H-pyrrolo[2,3-c]pyridin-4-yl)phenyl)ethanesulfonamide (ABBV-075); (2-cyclopropyl-5-(3,5-dimethyl-3H-113-isoxazol-4-yl)-1H-benzo[d]imidazol-7-yl)di(pyridin-2-yl) methanol (GS-5829, Alobresib); (S)-4-(6-(3,5-dimethylisoxazol-4-yl)-1-(1-(pyridin-2-yl)ethyl)-1H-pyrrolo[3,2-b]pyridin-3-yl)benzoic acid (PLX51107); methyl (S)-2-(4-(4-chlorophenyl)-2,3,9-trimethyl-6H-thieno[3,2-f][1,2,4]triazolo[4,3-a][1,4]diazepin-6-yl)acetate (MS417); TEN-010; ZEN003694; GSK2820151; FT-1101; olinone; compounds that interfere with binding of bromodomain-containing BET proteins to post-translationally modified histones (e.g., acetylated histones); compounds that disrupt formation of chromatin complexes critical for mRNA transcription, elongation, and splicing; compounds that compete with acetylated peptide binding (e.g., acetyl-lysine); compounds that suppress Cytosine-phosphate-Guanine (CpG) island negative genes (CGI-genes) (e.g., ABBV-075; ABBV-744; 2-((S)-4-(4-chlorophenyl)-2,3,9-trimethyl-6H-thieno[3,2-f][1,2,4]triazolo [4,3-a][1,4]diazepin-6-yl)-N-(4-(2-(2-(2-(2-((2-(2,6-dioxopiperidin-3-yl)-1,3-dioxo isoindolin-4-yl) amino)ethoxy)ethoxy)ethoxy) ethoxy)phenyl)acetamide (ARV-825); AZD 5153; 2-[[[4-(1,2-Dihydro-2-methyl-1-oxo-2,7-naphthyridin-4-yl)-2-6-dimethoxyphenyl]methyl]methyl amino]-N-[2-[2-[2-[[2-(2,6-dioxo-3-piperidinyl)-2,3-dihydro-1,3-dioxo-1H-isoindol-4-yl]amino] ethoxy]ethoxy]ethyl] acetamide dihydrochloride (dBRD9); MS645; PLX51107; CPI-203; I-BET 151; I-BET 762; MS417; OTX-015); and the like, or pharmaceutically acceptable enantiomers, diastereomers, racemates, and salts thereof. For example, the BET inhibitor may be tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof.

Another aspect may provide a method of treating a subject suffering from an age-related inflammatory disease in a subject by administering a therapeutically effective amount of tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof, to the subject in need thereof. These methods of treating described here may delay aging, delay the onset of age-related inflammatory diseases, and/or extend lifespan of a subject or cell (e.g., senescent cell). The lifespan may increase by 1% or more, by 90% or less, or by 1% - 50% (e.g., 5%, 10%, 11%, 12%, 13%, 14%, 15%, 20%, 25%, 30%, 35%, 40%, 45%). The age-related inflammatory disease described in any aspects herein may include, but is not limited to, Alzheimer’s disease, Parkinson’s, chronic inflammation, Rheumatoid arthritis, maculopathy atherosclerosis, diabetes, stroke, myocardial infarction, heart failure, hypertension, osteoarthritis, osteoporosis, sarcopenia, loss of bone marrow, idiopathic pulmonary fibrosis, degraded immune function, age-related macular degeneration, abnormal proliferative diseases, and disorders associated with a decrease in hormones (e.g., testosterone, estrogen, growth hormone, insulin-like growth factor I (IGF-I)) or reduced energy production. The methods may be used to treat a subject that is a mammal, such as but not limited to, a human, murine, canine, feline, or non-human primate.

A further aspect may provide a method of inhibiting CGI- gene misexpression in a cell by administering an effective amount of BET inhibitor to the cell. The cell of this aspect may include one or more cells, such as a senescent cell or aging cell. A plurality of these cells may include those that form a tissue and/or organ. Moreover, these cells may be present in an age-related inflammatory disease and/or subject suffering from an age-related inflammatory disease. The method of any of these aspects described herein may also inhibit nuclear lamina and/or heterochromatin disruption, inhibit CGI- misexpression associated organ failure, or inhibit age-associated degenerative changes (e.g., neurodegeneration, memory loss, osteoporosis, macular degeneration, hearing loss, heart disease, vascular disease, diabetes, metabolic syndrome, decreased organ function (e.g., lung, kidney), sarcopenia, frailty).

The invention provides methods and systems for screening potential BET inhibitor candidate substances in aging or age-related diseases, disorders, or conditions, as well as compositions containing any of these BET inhibitor candidate substances for use in the methods described here.

Other features and advantages of the invention will be apparent from the detailed description and from the claims.

### Definitions

Unless defined otherwise, all technical and scientific terms used herein have the meaning commonly understood by a person skilled in the art to which this invention belongs. All concentrations are in terms of percentage by weight of the specified component relative to the entire weight of the pharmaceutical composition, unless otherwise defined. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. Unless otherwise specified, the terms “first,” “second,” and the like, “primary,” “secondary,” and the like, do not denote any order, quantity, or importance, but rather are used to distinguish one element from another.

By “agent” is meant a peptide, nucleic acid molecule, or small molecule or chemical compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes at least a 5 % (e.g., 10%, 12%, 14%, 16%, 20%, 25%, 30%, 35%, 40%, 50%, 60%) or greater change in expression levels or 5%-50% (e.g., 10%-30%, 14%-25%) change in expression levels.

By “analog” is meant a molecule that is not identical but has analogous functional or structural features. For example, a polypeptide analog retains the biological activity of a corresponding naturally-occurring polypeptide, while having certain biochemical modifications that enhance the analog’s function relative to a naturally occurring polypeptide. Such biochemical modifications could increase the analog’s protease resistance, membrane permeability, or half-life, without altering, for example, ligand binding. An analog may include an unnatural amino acid.

By “CGIs” or “CpG islands” as used herein are DNA elements with high frequencies of Cytosine-phosphate-Guanine (CpG) dinucleotides and are typically associated with gene promoters and regulate promoters in subjects. In mammalian genomes, CpG islands usually extend for 300-3000 base pairs. They are located within and close to sites of about 40% of mammalian gene promoters.

In this disclosure, “comprises,” “comprising,” “containing,” and “having” and the like can have the meaning ascribed to them in U.S. Pat. law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant any molecule or composition that when linked to a molecule of interest (e.g., analyte, detector reagent, analog, or binding partner) renders the latter detectable, via spectroscopic, photochemical, biochemical, immunochemical, electrical, optical, or chemical means. For example, useful labels include enzymes (for example, as commonly used in an ELISA), enzyme substrates, radioactive isotopes, magnetic beads, metallic beads, colloidal particles (e.g., colloidal gold particles), chemiluminescent or fluorescent dyes, electron-dense reagents, enzymes, biotin, digoxigenin, or haptens, and the like. Methods for labeling and guidance in the choice of labels appropriate for various purposes are discussed, e.g., in Green, M. R., Hughes, H., Sambrook, J., & MacCallum, P. (2012). Molecular cloning: a laboratory manual. In Molecular cloning: a laboratory manual and Ausubel et al., Current Protocols in Molecular Biology. (1998). United States: John Wiley & Sons. The attachment of a compound (e.g., an antibody) to a label may be through covalent bonds, adsorption processes, hydrophobic and/or electrostatic bonds, as in chelates and the like, or combinations of these bonds and interactions and/or may involve a linking group.

By “disease” is meant any condition or disorder that damages or interferes with the normal function of a cell, tissue, or organ. As used herein, “disease” may be used interchangeably with disorder or condition. Non-limiting examples of diseases include aging or age-related disease, disorder, or conditions selected from neurodegenerating disease, myocardial infarction (i.e., heart attack), heart failure, atherosclerosis, hypertension, osteoarthritis, osteoporosis, sarcopenia, loss of bone marrow, cataract, multiple sclerosis, Sjögren’s syndrome, Rheumatoid arthritis, degraded immune function, diabetes, Idiopathic pulmonary fibrosis, and age-related macular degeneration, cerebellar infarction, stroke, Alzheimer’s disease, Parkinson’s disease, Huntington’s disease, tumorigenesis and malignant cancer development, chronic inflammation, maculopathy, inflammatory-related aging diseases, and disorders caused by the decline in testosterone, estrogen, growth hormone, IGF-I, or energy production.

The term “age-related disease” or “age-related disorder” refers to disorders or diseases where aging is a major risk factor, such as those categorized by: degenerative diseases, including neuron degenerating disease (e.g., Alzheimer’s, Parkinson’s, stroke), myocardial infarction, heart failure, atherosclerosis, hypertension, osteoarthritis, osteoporosis, sarcopenia, loss of bone marrow, rheumatoid arthritis, degraded immune function, diabetes, idiopathic pulmonary fibrosis, age-related macular degeneration; abnormal proliferative diseases (e.g., cancer); and disorders associated with a decrease in function, including a decrease in hormones (e.g., testosterone, estrogen, growth hormone, insulin-like growth factor I (IGF-I), reduced energy production, and the like. “Age-related inflammatory disease” or “age-related inflammatory disorders” refer to age-related diseases or disorders that are associated with inflammation or proinflammation. The term “proinflammation” refers to the promotion of inflammation via mediators that promote inflammation, such as but not limited to, cytokines, chemicals, vasoactive amines (e.g., histamine, serotonin), proteins or peptides (e.g., bradykinin), eicosanoids (e.g., thromboxanes, leukotrienes, prostaglandins). Non-limiting examples of age-related inflammatory diseases or disorders may include Alzheimer’s disease, atherosclerosis, heart disease, type II diabetes, and cancer.

By “anti-aging effect” means phenotypes comprising increased mitochondrial biogenesis and function, reduced reactive oxygen species (ROS) levels, extended life span of subjects or cells (such as senescent cells), post-mitotic cells (such as neuron cells), tissues, organs, organisms, prevented age-related disorders (e.g., tumorigenesis, malignant progression of cancers, cerebellar infarction, and myocardial infarction).

“Age-associated degenerative changes” as used herein, means changes resulting in, for example, neurodegeneration, memory loss, osteoporosis, macular degeneration, hearing loss, heart disease, vascular disease, diabetes, metabolic syndrome, decreased organ function (e.g., lung, kidney), sarcopenia, and frailty.

“Senescence” as used herein, means a cell cycle-arrested state in mitotic cells. This may be induced by, for example, telomere dysfunction (e.g., altering the maintenance, function, or structure of telomeres), DNA damage (e.g., due to oxidative stress and/or oxidative), cellular response to environmental damage or disease or immune response or genetic alteration of cells, or oncogene activation. In mammalian cells (e.g., human, murine), senescent cells are known to be arrested at the G0 phase, i.e., a non-dividing phase not of the cell cycle. Senescence or senescent cells means that cells show no increase in number or an increase in cell death and can exhibit β-galactosidase positive staining (e.g., senescence-associated beta-galactosidase (SA-β-gal)). Any mammalian cells that may undergo senescence are contemplated for use in the systems and methods described here. Senescent cells may be mammalian cells from humans 60 years old or older, while non-senescent cells may be from healthy humans less than 60 years old (e.g., 55 years old, 50 years old, 45 years old, 40 years old, 35 years old, 30 years old, 25 years old, 20 years old, 15 years old, 10 years old).

The term “effective amount” or “therapeutically effective amount” of an agent (e.g compounds having the structure of formula (I), etc.), as used herein, is that amount sufficient to effect beneficial or desired results, such as clinical results, and, as such, an “effective amount” depends upon the context in which it is being applied. In some embodiments, the compounds are administered in an effective amount for the treatment or prophylaxis of a disease disorder or condition. In another embodiment, in the context of administering an agent that is an anti-aging agent, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; delay or slowing the progress of the disease, disorder, or condition; amelioration or palliation of the disease, disorder, or condition (e.g., cancer, etc.); and remission (whether partial or total), whether detectable or undetectable, as compared to the response obtained without administration of the agent. The amount of BET inhibitor which constitutes an “effective amount” will vary depending on the BET inhibitor used, the severity of the disease, and the age and body weight of the subject to be treated, but can be determined routinely by a person of ordinary skill in the art having regard to known information and knowledge and to this disclosure.

A number of targets are useful for the development of highly specific drugs to treat a disease or disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, low complexity, and low cost.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain any portion of a polypeptide or nucleic acid molecule or sequence, such as but not limited to, 3, 4, 5, 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “BET inhibitor” is meant herein any agent that either directly or indirectly inhibits, blocks, or prevents bromodomain and extra terminal domain protein interactions or inhibits bromodomain function and/or activity. The BET inhibitors described here may inhibit the function and/or activity of any BET proteins, such as but not limited to, BRD2, BRD3, BRD4, and BRDT, or any targets thereof. In some instances, BET inhibitors may modulate transcriptional processes.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation is at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 5% (e.g., 10%, 25%, 50%, 75%, 100%.)

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least 16 amino acids (e.g., 20 amino acids, 25 amino acids, 35 amino acids, 50 amino acids, 100 amino acids). For nucleic acids, the length of the reference nucleic acid sequence will generally be at least 50 nucleotides (e.g., 60, 75, 100, 200, 300), and in some instances 100 nucleotides or 300 nucleotides or any integer thereabout or therebetween.

By “siRNA” is meant a double stranded RNA. In some instances, an siRNA is 10 or more nucleotides in length (e.g., 18, 19, 20, 21, 22, 23 or 24 nucleotides in length) and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than 750 mM NaCl and 75 mM trisodium citrate, less than 500 mM NaCl and 50 mM trisodium citrate, and less than 250 mM NaCl and 25 mM trisodium citrate. Low stringency hybridization can be obtained in the absence of organic solvent, e.g., formamide, while high stringency hybridization can be obtained in the presence of at least 35% formamide, and at least 50% formamide. Stringent temperature conditions will ordinarily include temperatures of at least 30° C., of at least 37° C., and of at least 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In one embodiment, hybridization will occur at 37° C. in 500 mM NaCl, 50 mM trisodium citrate, 1% SDS, 35% formamide, and 100 .mu.g/ml denatured salmon sperm DNA (ssDNA). In another embodiment, hybridization will occur at 42° C. in 250 mM NaCl, 25 mM trisodium citrate, 1% SDS, 50% formamide, and 200 µg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than 30 mM NaCl and 3 mM trisodium citrate, and less than 15 mM NaCl and 1.5 mM trisodium citrate. Stringent temperature conditions for the wash steps will ordinarily include a temperature of at least 25° C., of at least 42° C., and of at least 68° C. In one embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In yet a further embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60% (e.g., 75%, 80%, 85%, 90%, 95%, 97%, 99%) identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the term “subject” refers to any organism to which a composition and/or compound in accordance with the disclosure may be administered, e.g., for experimental, diagnostic, prophylactic, and/or therapeutic purposes. Typical subjects include any animal (e.g., mammals such as murine, canine, feline, non-human primates, and humans, etc.). A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

Ranges provided herein are understood to be shorthand for all of the values within the range. For example, a range of 1 to 50 is understood to include any number, combination of numbers, or subrange from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. Typically, the treatment of a disease, disorder, or condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition, such as an age-related inflammatory disease. The phrase “preventing an aging or age-related inflammatory disease” means reducing the incidences, delaying, or reversing the progression of and/or diseases or symptoms related to aging or age-related inflammatory diseases.

The term “pharmaceutical composition,” as used herein, represents a composition containing a compound or other candidate substance described herein formulated with a pharmaceutically acceptable excipient. The compound or age-related inflammatory disease inhibitor described herein may be an inhibitor of the bromodomain and extra terminal domain (BET) family of proteins or target thereof. The BET family may include but is not limited to, BRD2, BRD3, BRD4, and BRDT. The age-related inflammatory disease inhibitor may be an inhibitor of any one or more of the BET family, including but not limited to, BRD2, BRD3, BRD4, and BRDT. Non-limiting examples of BET inhibitors useful in the invention described herein include: tert-butyl (S)-2-(4-(4-chlorophenyl)-2,3,9-trimethyl-6H-thieno[3,2-f][1,2,4]triazolo[4,3-a][1,4]diazepin-6-yl)acetate (JQ1); (R)-7-(3,5-dimethyl isoxazol-4-yl)-8-methoxy-1-(1-(pyridin-2-yl)ethyl)-1,3-dihydro-2H-imidazo[4,5-c]quinolin-2-one (I-BET 151); (S)-2-(6-(4-chlorophenyl)-8-methoxy-1-methyl-4H-benzo[f][1,2,4]triazolo[4,3-a][1,4] diazepin-4-yl)-N-ethylacetamide (I-BET 762); (S)-2-(6-(4-chlorophenyl)-8-methoxy-1-methyl-4H-benzo[f][1,2,4]triazolo[4,3-a][1,4]diazepin-4-yl)-N-ethylacetamide (OTX-015); (S)-2-(4-(4-chloro phenyl)-2,3,9-trimethyl-6H-thieno[3,2-f][1,2,4]triazolo[4,3-a][1,4]diazepin-6-yl)acetamide (CPI-203); (S)-2-(6-(4-chlorophenyl)-1-methyl-4H-benzo[c]isoxazolo[4,5-e]azepin-4-yl)acetamide (CPI-0610); 2-(4-(2-hydroxyethoxy)-3,5-dimethylphenyl)-5,7-dimethoxyquinazolin-4(3H)-one (RVX-208); (Z)-4-(2-(2-amino-5-methyl-4-oxocyclohexa-2,5-dien-1-ylidene)hydrazineyl)-N-(pyridin-2-yl)benzene sulfonamide (MS436); 2-methoxy-N-(3-methyl-2-oxo-1,2,3,4-tetrahydroquinazolin-6-yl)benzene sulfonamide (PFI-1); N-ethyl-4-(2-(4-fluoro-2,6-dimethylphenoxy)-5-(2-hydroxypropan-2-yl)phenyl)-6-methyl-7-oxo-6,7-dihydro-1H-pyrrolo[2,3-c]pyridine-2-carboxamide (ABBV-744); 2-Morpholin-4-yl-8-phenylchromen-4-one (LY294002); (R)-4-(2-(4-(1-(3-methoxy-[1,2,4]triazolo[4,3-b]pyridazin-6-yl)piperidin-4-yl)phenoxy)ethyl)-1,3-dimethylpiperazin-2-one (AZD 5153); N,N′-(3,6,9,12,15,18,21-heptaoxatricosane-1,23-diyl)bis(2-((S)-4-(4-chlorophenyl)-2,3,9-trimethyl-6H-thieno[3,2-f][1,2,4]tri-azolo[4,3-a][1,4]diazepin-6-yl)acetamide) (MT-1); (S)-N,N′-(decane-1,10-diyl)bis(2-((6S)-4-(4-chlorophenyl)-2,3,9-trimethyl-6H-thieno[3,2-f] [1,2,4]triazolo[4,3-a][1,4]diazepin-6-yl)acetamide) (MS645); 2-(4-(2-(isopropylamino)ethoxy)-3,5-dimethylphenyl)-5,7-dimethoxyquinazolin-4(3H)-one (RVX2135); (S)-7,8-dimethoxy-N,4-dimethyl-1-(4-(4-methylpiperazin-1-yl)phenyl)-4,5-dihydro-3H-benzo[d][1,2]diazepine-3-carboxamide (BAY1238097); (S)-6-(3,5-dimethylisoxazol-4-yl)-3-(pyridin-2-yl)-3,4-dihydro-5-oxa-1,2a-diazaacenaphthylen-2(1H)-one (INCB054329); 6-(3-hydroxypropyl)-2-(1,3,6-trimethyl-2-oxo-2,3-dihydro-1H-benzo[d]imidazol-5-yl)-1H-benzo[de]isoquinoline-1,3(2H)-dione (BAY-299); (S)-2-(3-(1,4-dimethyl-1H-1,2,3-triazol-5-yl)-5-(phenyl(tetrahydro-2H-pyran-4-yl)methyl)-5H-pyrido[3,2-b]indol-7-yl)propan-2-ol (BMS-986158); N-(4-(2,4-difluorophenoxy)-3-(6-methyl-7-oxo-6,7-dihydro-1H-pyrrolo[2,3-c]pyridin-4-yl)phenyl)ethanesulfonamide (ABBV-075); (2-cyclopropyl-5-(3,5-dimethyl-3H-113-isoxazol-4-yl)-1H-benzo[d]imidazol-7-yl)di(pyridin-2-yl) methanol (GS-5829, Alobresib); (S)-4-(6-(3,5-dimethylisoxazol-4-yl)-1-(1-(pyridin-2-yl)ethyl)-1H-pyrrolo[3,2-b]pyridin-3-yl)benzoic acid (PLX51107); methyl (S)-2-(4-(4-chlorophenyl)-2,3,9-trimethyl-6H-thieno[3,2-f][1,2,4)triazolo[4,3-a][1,4]diazepin-6-yl)acetate (MS417); TEN-010; ZEN003694; GSK2820151; FT-1101; olinone; compounds that interfere with binding of bromodomain-containing BET proteins to post-translationally modified histones (e.g., acetylated histones); compounds that disrupt formation of chromatin complexes critical for mRNA transcription, elongation, and splicing; compounds that compete with acetylated peptide binding (e.g., acetyl-lysine); compounds that suppress Cytosine-phosphate-Guanine (CpG) island negative genes (CGI-genes) (e.g., ABBV-075; ABBV-744; 2-((S)-4-(4-chlorophenyl)-2,3,9-trimethyl-6H-thieno[3,2-f][1,2,4]triazolo [4,3-a][1,4]diazepin-6-yl)-N-(4-(2-(2-(2-(2-((2-(2,6-dioxopiperidin-3-yl)-1,3-dioxo isoindolin-4-yl) amino)ethoxy)ethoxy)ethoxy) ethoxy)phenyl)acetamide (ARV-825); AZD 5153; 2-[[[4-(1,2-Dihydro-2-methyl-1-oxo-2,7-naphthyridin-4-yl)-2-6-dimethoxyphenyl]methyl] methyl amino]-N-[2-[2-[2-[[2-(2,6-dioxo-3-piperidinyl)-2,3-dihydro-1,3-dioxo-1H-isoindol-4-yl]amino] ethoxy] ethoxy]ethyl] acetamide dihydrochloride (dBRD9); MS645; PLX51107; CPI-203; I-BET 151; I-BET 762; MS417; OTX-015); and the like, or pharmaceutically acceptable enantiomers, diastereomers, racemates, and salts thereof. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, etc.); for topical administration (e.g., as a cream, gel, lotion, or ointment); for intravenous administration (e.g., as a sterile solution free of particulate emboli and in a solvent system suitable for intravenous use); or in any other formulation described herein.

As used herein, the phrase “pharmaceutically acceptable” generally safe for ingestion or contact with biologic tissues at the levels employed. Pharmaceutically acceptable is used interchangeably with physiologically compatible. It will be understood that the pharmaceutical compositions of the disclosure include nutraceutical compositions (e.g., dietary supplements) unless otherwise specified.

Unit dosage forms, also referred to as unitary dosage forms, often denote those forms of medication supplied in a manner that does not require further weighing or measuring to provide the dosage (e.g., tablet, capsule, caplet, etc.). For example, a unit dosage form may refer to a physically discrete unit suitable as a unitary dosage for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, and gel cap. In certain embodiments, the compounds described herein, including crystallized forms, polymorphs, and solvates thereof, may be present in a unit dosage form.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Oral compositions that pass through the gastrointestinal (GI) tract can be formulated to overcome barriers, including but not limited to, acidic conditions, quick time for passage through the GI tract, active release variability, break down of enzymes, and the like. In some embodiments, carriers that may overcome such barriers may include hydroxypropyl methylcellulose phthalate (HPMCP) or thiolated HPMCP. Water, saline, aqueous dextrose, and glycols are examples of liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington’s Pharmaceutical Sciences by E. W. Martin. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer’s solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

The compounds described herein may be present as a pharmaceutically acceptable salt. Typically, salts are composed of a related number of cations and anions (at least one of which is formed from the compounds described herein) coupled together (e.g., the pairs may be bonded ionically, etc.) such that the salt is electrically neutral. Pharmaceutically acceptable salts may retain or have similar activity to the parent compound (e.g., an ED50 within 10%, etc.) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P.H. Stahl and C.G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

Compounds provided herein can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system). Atoms (other than H) on each side of a carbon- carbon double bond may be in an E (substituents are on opposite sides of the carbon- carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99%, or 99.9%) by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either stereoisomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Compounds provided herein can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system). Atoms (other than H) on each side of a carbon- carbon double bond may be in an E (substituents are on opposite sides of the carbon- carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99%, or 99.9%) by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either stereoisomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Solvates of the compounds described herein may be an aggregate of the compound or an ion of the compound with one or more solvents. Such solvents may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The crystalline form of the compounds described herein may refer to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

Any compositions or methods provided herein can be combined with one or more of any of the other compositions and methods provided herein.

### BRIEF DESCRIPTION OF THE DRAWINGS

FIG. 1A illustrates a nuclear architecture of chromatin in normal cells; and FIG. 1B a disorganization of chromatin architecture during aging. These contexts exhibit nuclear lamina/INM protein disruption, as well as global heterochromatin decondensation. FIG. 1C and FIG. 1D show epigenetic regulation in CGI+ and CGI- genes within normal nucleus. Only CGI- genes can form heterochromatin when they are transcriptionally inactive. PRC: Polycomb repressive complex. FIG. 1E provides CGI-guided models for gene localization within normal nucleus. Only CGI- genes can be associated with nuclear peripheral lamina forming heterochromatin. FIG. 1F shows that CGI-genes may be mislocalized/misexpressed in aged nuclei, as a result of disruption of the nuclear lamina (NL), inner nuclear membrane (INM) proteins, and heterochromatin.

FIG. 2 presents the overarching hypothesis that aging-mediated changes in chromatin architecture specifically disrupt regulation of CGI- genes.

FIG. 3 shows that normative aging causes CGI- gene misexpression in vivo. FIG. 3A and FIG. 3B provides an example of gene expression in young and old mouse kidneys. P-values are from Wilcoxon test. AUC: Area under curve. FIG. 3C presents data in 192 Diversity Outbred mice: a distribution shift where the right panel shows CGI- gene expression; net expression changes primarily showing CGI- gene expression under “UP”; and portions of differentially expressed (over 2-fold) genes, where for the “DN” expression from 0% to -20% shows CGI+ then primarily CGI- gene expression and for the “UP” expression from 0% to 50% shows CGI+ then primarily CGI- gene expression. All values were calculated via comparison with median expression in sex-matched young (6-month-old) kidneys. All samples were sorted by the distribution shift of all genes of FIG. 3B in each age group. FIG. 3D shows a Western blot analysis of heterochromatin protein/marks in the selected samples from FIG. 3B (Upper) and α-tubulin-normalized Western blot band intensities (Lower). *p < 0.05 (2-sample permutation). FIG. 3E illustrates immunofluorescence images of lamin B1 and H3K9me⅔ (left) and their intensities (right). ***p < 0.001. FIG. 3F provides urine microalbumin-to-creatinine ratios in unaffected and affected mice.

FIG. 4A provides a misexpression of CGI- genes in aged tissues. FIG. 4A presents cases are in non-regenerative/post-mitotic and FIG. 4B illustrates highly regenerative tissue/organs. Significance (distribution shift) and the directionality (det expression change) of gene expression changes in aged tissues vs. young are shown as violin plots. FIG. 4C shows that various known aging intervention can revert/suppress misexpression of CGI- genes. *P < 0.05, **P < 0.01, ***P < 0.001; n.s: not significant from one-tailed Student’s T-test. All data are from the comparison with young controls for CGI+ genes (upper) and CGI- genes (lower). Germ Layers - Endoderm: Liver, Small intestine; Mesoderm: Adipose, Bone, Kidney, Muscle, Heart, Whole blood, Bone marrow; Ectoderm: Brain, Spinal cord, Skin, Gingiva; Sex - F: female; M: male.

FIG. 5A shows the anticipated outcome of CGI-mediated gene segregation in aged nuclei. FIG. 5B provides spatial segregation between CGI+ (red) and CGI- (white) genes (FIG. 1E) visualized by multi-loci 3-D FISH. Arrows and brackets point to CGI+ genes. Scale bar: 5 µm.

FIG. 6 presents the misexpression of CGI- genes upon nuclear architecture/heterochromatin disruption. FIG. 6A illustrates gene expression changes upon nuclear lamina (lamin B receptor (Lbr) & lamin B1 (Lmnbl)) or heterochromatin (HP1α) disruption, as well as in progeria and cellular senescence. FIG. 6B shows an overlap among genes upregulated upon nuclear lamina/heterochromatin disruption, as well as in various aging contexts. #: data from immortalized cell cultures.

FIG. 7 provides a misexpression of CGI- genes in age-associated diseases. All data are from the comparison with normal tissues for CGI+ genes (upper) and CGI- genes (lower). *P < 0.05; **P < 0.01, ***P < 0.001 from one-tailed Student’s T-test.

FIG. 8 illustrates misexpression of tissue-specific genes during aging. Misexpression of tissue-specific genes in aged Diversity Outbred (“DO”) kidneys (FIG. 8A) or upon nuclear architecture disruption and in progeria/senescence (FIG. 8B). CGI+ genes protrude to the right of CGI- genes.

FIG. 9 shows an increased noise in CGI- gene transcription during aging.

FIG. 10 demonstrates a misexpression of secretory factors during aging. FIG. 10A provides GO:CC (Gene Ontology:Cellular Component) enrichment analysis of the entire CGI+ and CGI- genes. FIG. 10B presents expression of extracellular secretory factors in DO kidneys. Overlap between differentially expressed genes and GO:0005576 (extracellular region) are shown. Dotted lines show the boundaries of tubles. The functions (GOBP) from left to right: Cytokine & chemokine signaling (black); Protease activity (green); Extracellular matrix formation (dark blue); Growth factor activity (light blue); Other immune responses (grey). CGI+ genes protrude to the right of CGI- genes. FIG. 10C illustrates mRNA-FISH (RNA-scope) analysis of pro-inflammatory secretory factor (Lcn2; solid arrows; yellow) in DO kidneys. DNA staining (DAPI; blue) shown by brackets and histone 3 lysine 9 di-/tri-methylation (H3K9me⅔) staining FIG. 10D shows expression of extracellular secretory factors in nuclear architecture disruption. Overlap between differentially expressed genes and GO:0005576 are shown. The functions (GOBP) from left to right: Cytokine & chemokine signaling (black); Protease activity (green); Extracellular matrix formation (dark blue); Growth factor activity (light blue); Other immune responses (grey). CGI+ genes protrude to the right of CGI- genes. FIG. 10E provides plasma proteome changes during aging. Data are from reanalysis of published genome-wide plasma proteome profiling data.

FIG. 11 presents transcriptional regulation of CGI- genes. FIG. 11A illustrates CGI-guided models for transcriptional regulation. FIG. 11B shows binding target gene expression changes upon TF (ATF4) knockout (KO) (ATF^{-/-}) in mouse embryonic fibroblasts (MEFs), where the wild type (WT) curve is to the left of the ATF^{-/-}. (Han et al. Nat. Cell Biol. 15:481-490, 2013). FIG. 11C provides a knockdown (KD) of a coactivator (p300) (p300-KD) in epithelial breast cancer cells (MCF7), where the control (Con) curve is to the left of the p300-KD curve (Siam et al. RNA 27:213-824, 2019), that suppresses global CGI- gene expression.

FIG. 12 illustrates JQ1 treatment suppresses CGI- gene misexpression. FIGS. 12A-12B show gene expression changes upon JQ1 treatment. FIG. 12A shows the control (Con) curve to the left of the JQ1 treatment (bromodomain BRD inhibition) curve. FIG. 12B shows that JQ1 suppresses CGI- gene expression. All data are from the comparison of JQ1 treated with untreated controls for CGI+ genes (upper) and CGI- genes (lower) for each cell type. TAC: transverse aortic constriction. FIG. 12C provides overlap between genes upregulated upon nuclear architecture disruption or during aging versus down-regulated genes upon JQ1 treatment. FIG. 12D illustrates lifespan extension of C. elegans upon a one-time JQ1 treatment. Representative data from three blind tests with similar results are shown. The control (Con) curve is to the left of the JQ1 treatment curve from 12-19 days post treatment.

FIG. 13 illustrates JQ1 in vivo treatment data based on published RNA-seq data generated from mice by intraperitoneal injections of 50 mg/kg/day as indicated. All data are from the comparison with untreated controls for CGI+ genes (upper) and CGI- genes (lower) for each of *p < 0.05; **p < 0.01, ***p< 0.001. Sham operation; transverse aortic constriction (TAC); myocardial infarction (MI).

FIG. 14 provides that BET inhibitors (left side) other than JQ1 also suppress CGI- gene expression at least for the indicated cell lines/tissues, concentrations, and durations. All data are from the comparison with untreated controls for CGI+ genes (upper) and CGI- genes (lower) for each of *p < 0.05; **p < 0.01, ***p< 0.001; n.s: not significant.

### DETAILED DESCRIPTION OF THE INVENTION

The invention features pharmaceutical compositions comprising a bromodomain and extraterminal domain (BET) inhibitor, methods, and systems for screening candidate substances or compounds thereof, for the prevention and/or treatment of age-related diseases, conditions, or disorders to a subject in need thereof. The invention also provides pharmaceutical compositions and methods thereof for treating or preventing an aging or age-related disease, condition, or disorder comprising a BET inhibitor and a pharmaceutically acceptable carrier. Another method provides a cost-effective, as well as time-effective (e.g., rapid, high-throughput) method of screening potential candidate substances for preventing and/or treating age-related inflammatory diseases, disorders, and conditions, such as but not limited to, Alzheimer’s diseases, chronic inflammation, Rheumatoid arthritis, and maculopathy, or diseases, disorders, and conditions that target or are associated with BET proteins.

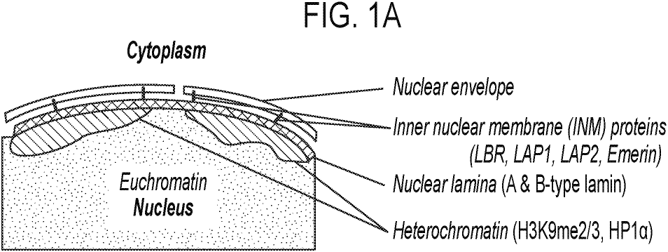

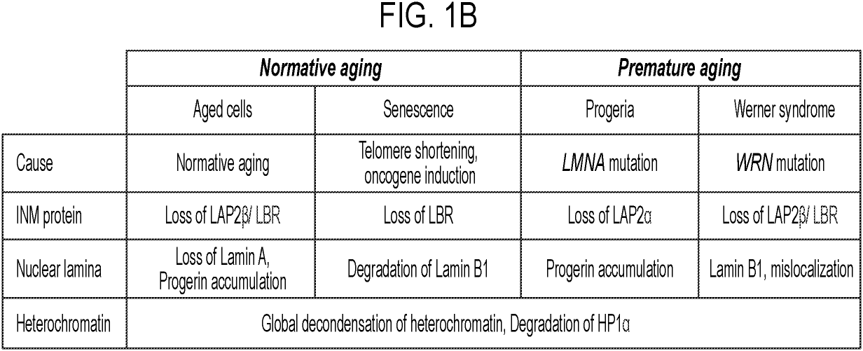

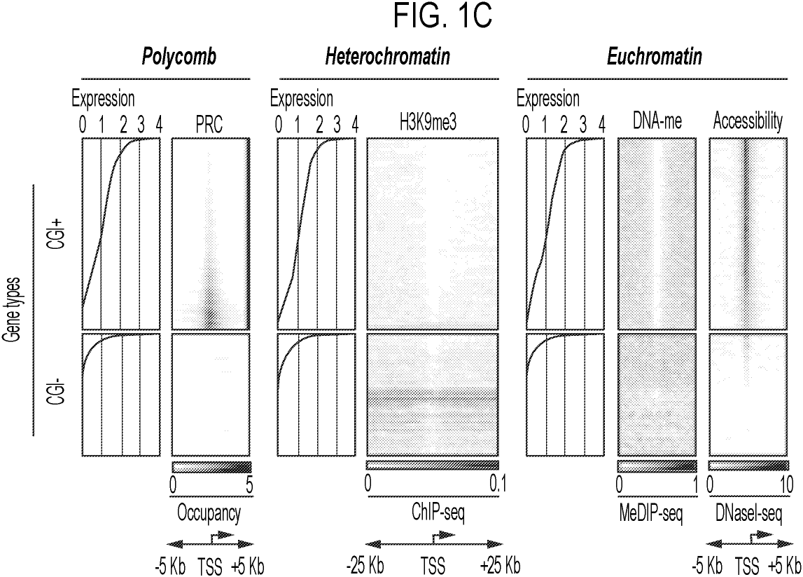

Post-translational modifications (PTMs) of histones are involved in regulation of gene expression and chromatin organization in eukaryotic cells. It was found that three-dimensional (3-D) chromatin architecture changes dynamically under a variety of conditions, including development, cell fate determination, and differentiation. Each cell type in the human body has a unique chromatin architecture that is optimized for the precise gene expression of that cell. Within the nucleus, chromatin is present as either condensed heterochromatin or loose euchromatin. Nuclear lamina is a dense, mesh-like protein layer, tethered to the nuclear envelope by inner nuclear membrane (INM) proteins (FIG. 1A). Nuclear lamina then physically anchors heterochromatin to the nuclear periphery. As a result of these series of interactions, heterochromatin is spatially separated from euchromatin located at the nuclear center. The precise formation and maintenance of heterochromatin is important for cell type-specific gene expression, and maintenance of cellular identity.

A growing body of evidence suggests that aging causes global disorganization of chromatin architecture. Specifically, disruption of the nuclear lamina and decondensation of its associated heterochromatin are commonly observed in various cellular aging contexts (FIG. 1B). Senescent and Werner syndrome cells exhibit global disruption of the nuclear lamina and INM components, lamin B1 and LBR (lamin B receptor), while progeria is directly caused by mutation of the nuclear lamina component lamin A. Cells from aged donors, including mesenchymal/ hematopoietic stem cells and fibroblasts, also exhibit global loss of INM (LAP2β and LBR) and nuclear lamina (lamin A) proteins. All of these aging contexts also result in global heterochromatin decondensation. Together, these results suggest that disorganization of nuclear chromatin architecture plays a major role in age-associated deterioration, as evidenced by the recent demonstration that forced maintenance of nuclear architecture can delay cellular senescence, in vitro. Although these shared changes in chromatin architecture have been reported for over two decades, the exact biological mechanisms by which such abnormal epigenetic/structural states lead to age-associated degenerative changes remain unclear.

### Compounds and Compositions

In one embodiment, the invention provides BET inhibitor compounds that are useful in methods described here, including but not limited to, treating a subject suffering from an age-related inflammatory disease in a subject and inhibiting BET protein activity or function in one or more senescent cells. The compounds described here, including their salts, may also include other solvents used for their crystallization or be in hydrate form. The compounds of the present invention may form solvates with pharmaceutically acceptable solvents including, for example, water. They may be formed inherently or by design. Embodiments of the invention embrace both solvated and unsolvated forms. The term “solvate” as used here refers to a complex of a compound, or pharmaceutically acceptable salts thereof, of the invention with one or more solvent molecules. These solvents or solvent molecules may be found and commonly used in the art, where the solvent is harmless to the recipient subject, e.g., water, ethanol, and the like. “Hydrate” is a term that refers to a complex where the solvent molecule is water. Accordingly, compounds of the invention may include salts, hydrates and solvates thereof, and form polymorphs. The compounds of the invention may include any BET inhibitors, agents that inhibit BET protein (either directly or indirectly), or agents that inhibit BET protein activity or function.

Non-limiting examples of compounds of the invention and BET inhibitors include tert-butyl (S)-2-(4-(4-chlorophenyl)-2,3,9-trimethyl-6H-thieno[3,2-f][1,2,4]triazolo[4,3-a][1,4]diazepin-6-yl)acetate (JQ1); (R)-7-(3,5-dimethylisoxazol-4-yl)-8-methoxy-1-(1-(pyridin-2-yl)ethyl)-1,3-dihydro-2H-imidazo[4,5-c]quinolin-2-one (I-BET 151); (S)-2-(6-(4-chlorophenyl)-8-methoxy-1-methyl-4H-benzo[f][1,2,4]triazolo [4,3-a][1,4]diazepin-4-yl)-N-ethylacetamide (I-BET 762); (S)-2-(6-(4-chlorophenyl)-8-methoxy-1-methyl-4H-benzo[f][1,2,4]triazolo[4,3-a][1,4]diazepin-4-yl)-N-ethyl acetamide (OTX-015); (S)-2-(4-(4-chlorophenyl)-2,3,9-trimethyl-6H-thieno[3,2-f][1,2,4]triazolo[4,3-a][1,4]diazepin-6-yl)acetamide (CPI-203); (S)-2-(6-(4-chlorophenyl)-1-methyl-4H-benzo[c]isoxazolo [4,5-e]azepin-4-yl)acetamide (CPI-0610); 2-(4-(2-hydroxyethoxy)-3,5-dimethylphenyl)-5,7-dimethoxyquinazolin-4(3H)-one (RVX-208); (Z)-4-(2-(2-amino-5-methyl-4-oxocyclohexa-2,5-dien-1-ylidene)hydrazineyl)-N-(pyridin-2-yl)benzenesulfonamide (MS436); 2-methoxy-N-(3-methyl-2-oxo-1,2,3,4-tetrahydroquinazolin-6-yl)benzenesulfonamide (PFI-1); N-ethyl-4-(2-(4-fluoro-2,6-dimethylphenoxy)-5-(2-hydroxypropan-2-yl)phenyl)-6-methyl-7-oxo-6,7-dihydro-1H-pyrrolo[2,3-c]pyridine-2-carboxamide (ABBV-744); 2-Morpholin-4-yl-8-phenylchromen-4-one (LY294002); (R)-4-(2-(4-(1-(3-methoxy-[1,2,4]triazolo[4,3-b]pyridazin-6-yl)piperidin-4-yl)phenoxy) ethyl)-1,3-dimethylpiperazin-2-one (AZD 5153); N,N′-(3,6,9,12,15,18,21-heptaoxatricosane-1,23-diyl)bis(2-((S)-4-(4-chlorophenyl)-2,3,9-trimethyl-6H-thieno[3,2-f][1,2,4] triazolo[4,3-a][1,4]diazepin-6-yl)acetamide) (MT-1); (S)-N,N′-(decane-1,10-diyl)bis(2-((6S)-4-(4-chlorophenyl)-2,3,9-trimethyl-6H-thieno[3,2-f] [1,2,4]triazolo[4,3-a][1,4]diazepin-6-yl)acetamide) (MS645); 2-(4-(2-(isopropylamino) ethoxy)-3,5-dimethylphenyl)-5,7-dimethoxyquinazolin4(3H)-one (RVX2135); (S)-7,8-dimethoxy-N,4-dimethyl-1-(4-(4-methylpiperazin-1-yl)phenyl)-4,5-dihydro-3H-benzo[d][1,2] diazepine-3-carboxamide (BAY1238097); (S)-6-(3,5-dimethylisoxazol-4-yl)-3-(pyridin-2-yl)-3,4-dihydro-5-oxa-1,2a-diazaacenaphthylen-2(1H)-one (INCB054329); 6-(3-hydroxypropyl)-2-(1,3,6-trimethyl-2-oxo-2,3-dihydro-1H-benzo[d]imidazol-5-yl)-1H-benzo[de]isoquinoline-1,3(2H)-dione (BAY-299); (S)-2-(3-(1,4-dimethyl-1H-1,2,3-triazol-5-yl)-5-(phenyl(tetrahydro-2H-pyran-4-yl)methyl)-5H-pyrido[3,2-b]indol-7-yl)propan-2-ol (BMS-986158); N-(4-(2,4-difluorophenoxy)-3-(6-methyl-7-oxo-6,7-dihydro-1H-pyrrolo[2,3-c]pyridin-4-yl)phenyl)ethanesulfonamide (ABBV-075); (2-cyclopropyl-5-(3,5-dimethyl-3H-113-isoxazol-4-yl)-1H-benzo[d]imidazol-7-yl)di(pyridin-2-yl) methanol (GS-5829, Alobresib); (S)-4-(6-(3,5-dimethylisoxazol-4-yl)-1-(1-(pyridin-2-yl)ethyl)-1H-pyrrolo[3,2-b]pyridin-3-yl)benzoic acid (PLX51107); methyl (S)-2-(4-(4-chlorophenyl)-2,3,9-trimethyl-6H-thieno[3,2-f][1,2,4]triazolo[4,3-a][1,4]diazepin-6-yl)acetate (MS417); TEN-010; ZEN003694; GSK2820151; FT-1101; olinone; compounds that interfere with binding of bromodomain-containing BET proteins to post-translationally modified histones (e.g., acetylated histones); compounds that disrupt formation of chromatin complexes critical for mRNA transcription, elongation, and splicing; compounds that compete with acetylated peptide binding (e.g., acetyl-lysine); compounds that suppress Cytosine-phosphate-Guanine (CpG) island negative genes (CGI- genes) (e.g., ABBV-075; ABBV-744; 2-((S)-4-(4-chlorophenyl)-2,3,9-trimethyl-6H-thieno[3,2-f][1,2,4]triazolo [4,3-a][1,4]diazepin-6-yl)-N-(4-(2-(2-(2-(2-((2-(2,6-dioxopiperidin-3-yl)-1,3-dioxoisoindolin-4-yl)amino)ethoxy)ethoxy)ethoxy) ethoxy)phenyl)acetamide (ARV-825); AZD 5153; 2-[[[4-(1,2-Dihydro-2-methyl-1-oxo-2,7-naphthyridin-4-yl)-2-6-dimethoxyphenyl]methyl]methylamino]-N-[2-[2-[2-[[2-(2,6-dioxo-3-piperidinyl)-2,3-dihydro-1,3-dioxo-1H-isoindol-4-yl]amino]ethoxy]ethoxy]ethyl] acetamide dihydro chloride (dBRD9); MS645; PLX51107; CPI-203; I-BET 151; I-BET 762; MS417; OTX-015); and the like, or pharmaceutically acceptable enantiomers, diastereomers, racemates, and salts thereof.

In some embodiments, a BET inhibitor of the disclosure may be structurally similar to JQ1 and similarly binds to BRD4 at various positions, including but not limited to, tryptophan at position 81 (Trp-81), proline at position 82 (Pro-82), tyrosine at position 97 (Tyr-97), asparagine at position 140 (Asn-140), and methionine at position 149 (Met-149), while having limited binding to, but not limited to, leucine at position 94 (Leu-94), tyrosine at position 139 (Tyr-139), aspartic acid at position 144 (Asp-144), aspartic acid at position 145 (Asp-145), and isoleucine at position 146 (Ile-146), which is described by Jung et al. (JBC 289(13):9304-9319, 2014) and incorporated by reference in its entirety. Some embodiments may be directed to a BET inhibitor structurally similar to JQ1 that binds mutated BRD4, where leucine at position 94 and aspartic acid at position 145 are each modified to alanine. See, Jung et al. for characterization of exemplary BET inhibitors. For example, the BET inhibitor may be tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof. In some embodiments, the BET inhibitor candidate substance may be tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof.

Compositions or pharmaceutical compositions described here may comprise one or more compounds and/or BET inhibitors described here, or a pharmaceutically acceptable salt thereof, and pharmaceutically acceptable carrier. The pharmaceutical compositions of the disclosure may be formulated for routes of administration such as but not limited to oral, topical, buccal, parenteral (e.g., subcutaneous, intramuscular, intradermal, intravenous), and rectal. The most suitable form of administration in any given case will depend on the degree and severity of the condition being treated and on the nature of the particular BET inhibitor compound being used.

The pharmaceutical compositions may be formulated such that they are suitable for oral administration in individual units including, for example, tablets, capsules, caplets, cachets, lozenges, cachets, dragées, solutions, suspensions, gels, and syrups. Each pharmaceutical composition may contain a pre-determined amount of a compound of the disclosure as powder or granules; as a solution or a suspension in an aqueous or non-aqueous liquid; or as an oil-in-water or water-in-oil emulsion. As indicated, such formulations may be prepared by any suitable pharmaceutical method, including but not limited to, the association of at least one compound of the present disclosure as an active compound and a carrier and/or excipient. The carrier will be compatible with the active compound and other ingredients of the formulation and not be harmful to the subject recipient. The carrier may be a solid or a liquid, or both, and may be formulated with at least one compound described herein as the active compound in a unit-dose formulation. Some non-toxic solid carriers, such as those for solid compositions, may include, but are not limited to, glucose, sucrose, magnesium carbonate, mannitol, lactose, starch, magnesium stearate, sodium saccharin, talc, cellulose, and the like, where the carriers are pharmaceutically acceptable.

In some embodiments, for liquid pharmaceutical compositions, an active compound of the disclosure may be prepared by dissolving or dispersing at least one of the active compounds and optionally a pharmaceutical adjuvant in a carrier or excipient, such as, for example, water, saline, aqueous dextrose, glycerol, ethanol, and the like, thereby forming a solution or suspension. Exemplary formulations may be prepared by admixing at least one active compound and a liquid or solid carrier finely divided, or both, in a uniform manner, and then shaping the product as needed. In one embodiment, a tablet may be prepared by compressing or molding a powder or granules of at least one compound as described here, which may be optionally combined with one or more additional ingredients. For example, an appropriate device or machine may be used in compressing at least one active compound, such as a powder or granules, and optionally mixing the active compound with another ingredient, e.g., binder, lubricant, inert diluent, surface active, dispersing agent, to form tablets. Tablets may also be made by molding, for example, a powdered form of at least one compound described here, by moistening the active compound with an inert liquid diluent. Non-limiting examples of excipients include fillers, extenders, diluents, wetting agents, solvents, emulsifiers, preservatives, flavors, absorption enhancers, sustained-release matrices, and coloring agents.

In some embodiments, a pharmaceutical composition may contain an effective amount of a compound and/or BET inhibitor disclosed here, where the amount is sufficient to inhibit BET protein activity and/or function in a senescent cell or treat a subject suffering from an age-related inflammatory disease or symptom thereof. One embodiment may be directed to a pharmaceutical composition containing 0.001 % to 95% (e.g., 0.01 % to 90%, 0.1 % to 85%, 1% to 80%, 5% to 75%, 10% to 70%) by weight of the one active compound or BET inhibitor, or any therapeutically effective amount of the active compound or BET inhibitor, relative to the weight of the pharmaceutical composition. In other embodiments, the pharmaceutical composition may contain 0.001% or greater (e.g., 0.01%, 0.1%, 1%, 5%, 10%, 20%, 50%, 70%, 75%, 80%, 85%, 90%, 95%) by weight of the active compound or BET inhibitor relative to the weight of the pharmaceutical composition. Yet another embodiment may provide a pharmaceutical composition containing 98% or less (e.g., 95%, 90%, 85%, 80%, 75%, 70%, 65%, 50%, 20%, 15%, 10%, 5%, 1%, 0.5%, 0.05%) by weight of the active compound or BET inhibitor relative to the weight of the pharmaceutical composition. In some embodiments, other pharmacologically active substances may also be present including other compounds. The pharmaceutical compositions or formulations disclosed here may be prepared by any of well-known pharmaceutical and formulation techniques that may include admixing the ingredients or components described here.

The amount of an active BET inhibitor and/or compound for administration may depend on the subject being treated, the subject’s weight, the administration route, and as determined by the prescribing physician. For example, an encapsulated compound at a perceived dosage of ~1 pg to ~1000 mg (e.g., 2 pg — 800 mg; 20 pg — 80 mg; 200 pg — 8 mg; 2 ng — 800 µg; 20 ng — 80 µg; 200 ng — 8 µg); 1 pg or greater (e.g., 10 pg; 100 pg; 1 ng; 10 ng; 100 ng; 1 µg; 100 µg; 1 mg; 10 mg; 100 mg; 1000 mg); 1000 mg or less (e.g., 500 mg; 50 mg; 5 mg; 500 µg; 50 µg; 5 µg; 500 ng; 50 ng; 5 ng; 500 pg; 50 pg; 5 pg) may involve daily or semi-daily administration. In some embodiments, intermittent administration of a dose of the encapsulated compound and/or BET inhibitor may be employed (e.g., a weekly, monthly, or yearly basis). In some embodiments, since encapsulation facilitates accessibility to an action site allowing administration of the active ingredients simultaneously, a synergistic effect may be produced. Physicians can readily determine optimal dosages and can readily modify administration or dosing schedules to achieve such dosages in accordance with standard dosing regimens.

A therapeutically effective amount of a disclosed BET inhibitor or composition comprising the BET inhibitor as described here, may be determined by the therapeutic effectiveness of the BET inhibitor. Depending on the subject, the severity of the condition being treated, and the BET inhibitor being used will determine the dosages. Some embodiments encompass a therapeutically effective amount of a BET inhibitor, such as one of those described here, sufficient to result in a maximal plasma concentration. Initial doses may be determined, for example, by animal testing and dose titrations for human administration as tested in accordance to accepted practices.

Standard pharmaceutical procedures in vitro and in vivo may be used to determine toxicity and therapeutic efficacy, for example, in calculating the LD₅₀ (i.e., median lethal dose to achieve 50% mortality from toxicity); the ED₅₀ (i.e., median effective dose to achieve 50% of the desired response in 50% of the population); and EC₅₀ (i.e., half maximum effective concentration of a drug at which 50% of its maximum response is observed). The dose ratio between toxic and therapeutic effects is the therapeutic index, which can be expressed as the ratio LD₅₀/ED₅₀. A safer desirable BET inhibitor exhibits a higher therapeutic index. In some embodiments, a BET inhibitor of the disclosure may have an LD₅₀ of 0.1 µM or greater (e.g., 0.2 µM; 0.3 µM; 0.4 µM; 0.5 µM; 0.6 µM; 0.7 µM; 0.8 µM; 0.9 µM; 1 µM); 1 µM or less (e.g., 0.95 µM; 0.85 µM; 0.75 µM; 0.65 µM; 0.55 µM; 0.45 µM; 0.35 µM; 0.25 µM; 0.15 µM); or 0.1 µM - 1 µM (e.g., 0.125 µM - 0.975 µM; 0.175 µM - 0.925 µM; 0.225 µM - 0.875 µM; 0.275 µM - 0.825 µM; 0.325 µM - 0.775 µM; 0.375 µM - 0.725 µM; 0.425 µM -0.675 µM; 0.475 µM - 0.625 µM; 0.525 µM - 0.575 µM; 0.228 µM - 0.46 µM; 0.344 µM).

Various embodiments may be directed to a BET inhibitor of the disclosure having an ED₅₀ or an EC₅₀ of 1 nM or greater (e.g., 2 nM; 4 nM; 6 nM; 8 nM; 10 nM; 20 nM; 40 nM; 60 nM; 80 nM; 100 nM; 120 nM; 140 nM; 160 nM; 180 nM; 200 nM; 220 nM; 240 nM; 260 nM; 280 nM; 300 nM; 320 nM; 340 nM; 360 nM; 380 nM; 400 nM; 420 nM; 440 nM; 460 nM; 480 nM; 500 nM; 520 nM; 540 nM; 560 nM; 580 nM; 600 nM; 620 nM; 640 nM; 660 nM; 680 nM; 700 nM; 720 nM; 740 nM; 760 nM; 780 nM; 800 nM; 820 nM; 840 nM; 860 nM; 880 nM; 900 nM; 920 nM; 940 nM; 960 nM; 980 nM; 1000 nM; 1020 nM; 1040 nM; 1060 nM; 1080 nM; 1100 nM; 1120 nM; 1140 nM; 1160 nM; 1180 nM; 1200 nM; 1220 nM; 1240 nM; 1260 nM; 1280 nM; 1300 nM; 1320 nM; 1340 nM; 1360 nM; 1380 nM; 1400 nM; 1420 nM; 1440 nM; 1460 nM; 1480 nM; 1500 nM; 1600 nM; 1700 nM; 1800 nM; 1900 nM; 2000 nM); 2000 nM or less (e.g., 1950 nM; 1850 nM; 1750 nM; 1650 nM; 1550 nM; 1450 nM; 1350 nM; 1250 nM; 1150 nM; 1050 nM; 950 nM; 850 nM; 750 nM; 650 nM; 550 nM; 450 nM; 350 nM; 250 nM; 150 nM; 50 nM; 25 nM; 15 nM; 5 nM); or 1 nM - 2000 nM (e.g., 3 nM -1990 nM; 7 nM - 1970 nM; 9 nM - 1930 nM; 13 nM - 1890 nM; 17 nM - 1870 nM; 19 nM - 1830 nM; 23 nM - 1790 nM; 27 nM - 1770 nM; 29 nM - 1730 nM; 33 nM - 1690 nM; 37 nM - 1670 nM; 39 nM - 1630 nM; 43 nM - 1590 nM; 47 nM - 1570 nM; 43 nM - 1530 nM; 47 nM - 1490 nM; 49 nM - 1470 nM; 53 nM - 1430 nM; 57 nM - 1390 nM; 59 nM - 1370 nM; 63 nM - 1330 nM; 67 nM - 1290 nM; 69 nM - 1270 nM; 73 nM - 1230 nM; 77 nM - 1190 nM; 79 nM - 1170 nM; 83 nM -1130 nM; 87 nM - 1090 nM; 93 nM - 1070 nM; 97 nM - 1030 nM; 99 nM - 990 nM; 103 nM - 970 nM; 107 nM - 930 nM; 109 nM - 890 nM; 113 nM - 870 nM; 117 nM - 830 nM; 119 nM - 790 nM; 123 nM - 770 nM; 127 nM - 730 nM; 129 nM - 690 nM; 133 nM - 670 nM; 137 nM - 630 nM; 139 nM - 590 nM; 143 nM - 570 nM; 147 nM - 530 nM; 149 nM - 490 nM; 153 nM - 470 nM; 157 nM - 430 nM; 159 nM - 390 nM; 163 nM - 370 nM; 167 nM - 330 nM; 169 nM - 290 nM; 173 nM -270 nM; 177 nM - 230 nM; 179 nM - 190 nM; 283 nM - 1923 nM; 500 nM).

In some embodiments, the toxic dose low (TDLo) is the lowest dose of a substance, such as a BET inhibitor, introduced by any route, other than inhalation, over any given period of time and reported to produce any toxic effect in humans or to produce tumorigenic, reproductive, or multiple dose effects in animals. Non-limiting TDLo values may include 1 mg/kg or greater (e.g., 5 mg/kg; 10 mg/kg; 50 mg/kg; 100 mg/kg; 200 mg/kg; 250 mg/kg; 400 mg/kg; 450 mg/kg; 600 mg/kg; 650 mg/kg; 800 mg/kg; 850 mg/kg); 1000 mg/kg or less (e.g., 950 mg/kg; 900 mg/kg; 750 mg/kg; 700 mg/kg; 550 mg/kg; 500 mg/kg; 350 mg/kg; 300 mg/kg; 150 mg/kg; 100 mg/kg; 25 mg/kg; 20 mg/kg); or 1 mg/kg - 1000 mg/kg (e.g., 3 mg/kg - 980 mg/kg; 7 mg/kg - 960 mg/kg; 9 mg/kg - 940 mg/kg; 11 mg/kg -920 mg/kg; 13 mg/kg - 880 mg/kg; 17 mg/kg - 860 mg/kg; 19 mg/kg - 840 mg/kg; 30 mg/kg - 820 mg/kg; 35 mg/kg - 780 mg/kg; 40 mg/kg - 760 mg/kg; 45 mg/kg - 740 mg/kg; 50 mg/kg - 720 mg/kg; 55 mg/kg - 680 mg/kg; 60 mg/kg - 660 mg/kg; 65 mg/kg - 640 mg/kg; 70 mg/kg - 620 mg/kg; 75 mg/kg - 580 mg/kg; 80 mg/kg - 560 mg/kg; 85 mg/kg - 540 mg/kg; 90 mg/kg - 520 mg/kg; 95 mg/kg - 480 mg/kg; 100 mg/kg - 460 mg/kg; 150 mg/kg - 440 mg/kg; 200 mg/kg - 420 mg/kg; 250 mg/kg - 380 mg/kg; 300 mg/kg - 360 mg/kg). Exemplary BET inhibitor (e.g., JQ1) LD₅₀, ED₅₀, and EC₅₀ values in various cells are described in Pulikkan et al. (Cell 174:172-186, 2018); Čančer et al. (Cell Death and Disease 10:881, 2019); Deng et al. (Translational Oncology 11(5):1147-1154, 2018); Cayman Chemical, Safety Data Sheet acc. To OSHA HCS, Jun. 25, 2021, which are all incorporated by reference in their entirety.

Other embodiments of the disclosure may include buccal or sub-lingual pharmaceutical compositions or formulations, including, for example, lozenges or pastilles containing at least one compound of the present disclosure. For example, the lozenge may comprise a compound or BET inhibitor as described here in a base (e.g., sucrose and acacia or tragacanth) and the pastille may contain a compound or BET inhibitor in an inert base (e.g., sucrose and acacia; gelatin and glycerin). Non-limiting examples of additional ingredients suitable for inclusion in a pharmaceutical composition of the disclosure include diluents, excipients, lubricants, binders, disintegrants, absorbents, colorants, flavors, and sweeteners. More specifically, exemplary additional ingredients include lactose, dextrose, sucrose, mannitol, sorbitol, cellulose, glycine, silica, talcum, stearic acid, magnesium or calcium salt and/or polyethyleneglycol, magnesium aluminum silicate, starch paste, gelatin, tragacanth, methylcellulose, sodium carboxymethylcellulose, hydroxypropyl methylcellulose phthalate (HPMCP), thiolated HPMCP (T-HPMCP), polyvinylpyrrolidone, starch, calcium carbonate, sodium carbonate, lactose, calcium phosphate, sodium phosphate, agar, alginic acid, sodium salt, effervescent mixtures, acacia. In some instances, the oral pharmaceutical compositions may be a tablet that is either uncoated or coated to delay release of the active compound, delay disintegration and/or absorption in the gastrointestinal tract of a subject, in order to provide sustained release of the active compound over a period of time.

Other injectable pharmaceutical compositions may contain the active compound in an aqueous isotonic solution or suspension, fatty emulsion, preserving agent, stabilizing agent, wetting agent, emulsifying agent, and the like. For example, the injectable composition may contain 0.001% to 90% (e.g., 0.01% to 85%, 0.1% to 80%, 1% to 50%) of the active compound or BET inhibitor.

Another embodiment may be directed to a topical pharmaceutical composition, including intranasal, comprising an active compound and a carrier (e.g., aqueous solution, suspension, ointment, cream, gel, aerosol) appropriate for topical use. Topical pharmaceutical compositions of the disclosure may also contain, for example, solubilizers, stabilizers, buffers, preservatives, tonicity enhancers, or combinations thereof. Formulations may be prepared as a powder for use in an inhaler or an aerosol spray as distributed from an atomizer, nebulizer, pump, spray, or pressurized container and contain an effective amount of an active compound or BET inhibitor as described here.

A further embodiment may include transdermal pharmaceutical compositions containing an effective amount of an active compound or BET inhibitor described here with a pharmaceutically acceptable carrier that is suitable for transdermal formulations that enable the active compound to pass through the skin of the subject, where passage is through at least the outermost epidermis layer.

The compounds or BET inhibitors, or salts thereof, of the disclosure may be used in the treatment of diseases such as age-related inflammatory diseases. Non-limiting age-related inflammatory diseases may include Alzheimer’s disease, atherosclerosis, heart disease, type II diabetes, and cancer.

The pharmaceutical composition in some embodiments of the disclosure may be a unit dosage of, for example, 0.5 mg -1000 mg (e.g., 1 mg - 500 mg, 1 mg - 250 mg, 1 mg - 150 mg, 1 mg - 100 mg, 1 mg - 50 mg) of active ingredient, where the subject may weigh 50 kg - 75 kg (or 110 lbs - 165 lbs). A therapeutically effective dosage of a compound described here or pharmaceutical compositions comprising the compound thereof, where the compound is a BET inhibitor, may depend on the species of the subject, the gender, the body weight, age, the disorder or disease, or the severity thereof being treated. A medical professional, physician, clinician, or veterinarian of ordinary skill can readily determine the effective amount of each of the active ingredients necessary to prevent, treat or inhibit the progress of the age-related inflammatory disease.

In some embodiments, the pharmaceutical composition containing an active compound, such as a BET inhibitor, as described here, and a pharmaceutically acceptable carrier may be administered in combination with another therapeutic agent, i.e., simultaneously, separately, before, after, in a fixed dosage pharmaceutical composition that contains the active compound and the therapeutic agent, or by different routes of administration. Non-limiting examples of a therapeutic agent may include a chemical compound, peptide, antibody, antibody fragment, or nucleic acid (e.g., DNA, RNA), where the therapeutic agent may be therapeutically active or enhance the therapeutic activity when administered to a subject in combination with a compound of the disclosure.

A further embodiment may be directed to a preparation of a medicament for the treatment of and/or protection against aging or age-related inflammatory diseases, or symptoms thereof, where the medicament contains a BET inhibitor or a candidate substance that acts or functions as a BET inhibitor, either directly or indirectly.

### Methods

An embodiment of the invention may be directed to a method of treating a subject suffering from an age-related inflammatory disease in a subject by administering a therapeutically effective amount of a Bromodomain and ExtraTerminal (BET) domain inhibitor (or BET inhibitor, used interchangeably here) to the subject in need thereof, by any of the routes of administration described here (e.g., oral, injection). Another embodiment may provide a method of treating a subject suffering from an age-related inflammatory disease in a subject by administering a therapeutically effective amount of a BET inhibitor, such as but not limited to, tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof, to the subject in need thereof. Various embodiments of the invention may utilize a BET inhibitor or a plurality of BET inhibitors selected from, but not limited to, tert-butyl (S)-2-(4-(4-chlorophenyl)-2,3,9-trimethyl-6H-thieno[3,2-f][1,2,4]triazolo[4,3-a][1,4]diazepin-6-yl)acetate (JQ1); (R)-7-(3,5-dimethylisoxazol-4-yl)-8-methoxy -1- (1-(pyridin-2-yl)ethyl)-1,3-dihydro-2H-imidazo[4,5-c]quinolin-2-one (I-BET 151); (S)-2-(6-(4-chlorophenyl)-8-methoxy-1-methyl-4H-benzo[f][1,2,4]triazolo [4,3-a][1,4]diazepin-4-yl)-N-ethyl acetamide (I-BET 762); (S)-2-(6-(4-chlorophenyl)-8-methoxy-1-methyl-4H-benzo[f][1,2,4] triazolo [4,3-a][1,4]diazepin-4-yl)-N-ethylacetamide (OTX-015); (S)-2-(4-(4-chlorophenyl)-2,3,9-trimethyl-6H-thieno[3,2-f][1,2,4]triazolo[4,3-a][1,4]diazepin-6-yl)acetamide (CPI-203); (S)-2-(6-(4-chlorophenyl)-1-methyl-4H-benzo[c]isoxazolo[4,5-e]azepin-4-yl)acetamide (CPI-0610); 2-(4-(2-hydroxyethoxy)-3,5-dimethylphenyl)-5,7-dimethoxyquinazolin-4(3H)-one (RVX-208); (Z)-4-(2-(2-amino-5-methyl-4-oxocyclohexa-2,5-dien-1-ylidene)hydrazineyl)-N-(pyridin-2-yl)benzenesulfonamide (MS436); 2-methoxy-N-(3-methyl-2-oxo-1,2,3,4-tetrahydroquinazolin-6-yl)benzenesulfon amide (PFI-1); N-ethyl-4-(2-(4-fluoro-2,6-dimethylphenoxy)-5-(2-hydroxypropan-2-yl)phenyl)-6-methyl-7-oxo-6,7-dihydro-1H-pyrrolo[2,3-c]pyridine-2-carboxamide (ABBV-744); 2-Morpholin-4-yl-8-phenylchromen-4-one (LY294002); (R)-4-(2-(4-(1-(3-methoxy-[1,2,4]triazolo[4,3-b]pyridazin-6-yl)piperidin-4-yl)phenoxy) ethyl)-1,3-dimethylpiperazin-2-one (AZD 5153); N,N′-(3,6,9,12,15,18,21-heptaoxatricosane-l,23-diyl)bis(2-((S)-4-(4-chlorophenyl)-2,3,9-trimethyl-6H-thieno[3,2-f][1,2,4] triazolo[4,3-a][1,4] diazepin-6-yl) acetamide) (MT-1); (S)-N,N′-(decane-1,10-diyl)bis(2-((6S)-4-(4-chlorophenyl)-2,3,9-trimethyl-6H-thieno[3,2-f] [1,2,4]triazolo[4,3-a][1,4]diazepin-6-yl)acetamide) (MS645); 2-(4-(2-(isopropylamino)ethoxy)-3,5-dimethylphenyl)-5,7-dimethoxyquinazolin4(3H)-one (RVX2135); (S)-7,8-dimethoxy-N,4-dimethyl-1-(4-(4-methylpiperazin-1-yl)phenyl)-4,5-dihydro-3H-benzo[d] [1,2]diazepine-3-carboxamide (BAY1238097); (S)-6-(3,5-dimethylisoxazol-4-yl)-3-(pyridin-2-yl)-3,4-dihydro-5-oxa-1,2a-diazaacenaphthylen-2(1H)-one (INCB054329); 6-(3-hydroxy propyl)-2-(1,3,6-trimethyl-2-oxo-2,3-dihydro-1H-benzo[d]imidazol-5-yl)-1H-benzo[de]isoquinoline-1,3(2H)-dione (BAY-299); (S)-2-(3-(1,4-dimethyl-1H-1,2,3-triazol-5-yl)-5-(phenyl(tetrahydro-2H-pyran-4-yl)methyl)-5H-pyrido[3,2-b]indol-7-yl)propan-2-ol (BMS-986158); N-(4-(2,4-difluoro phenoxy)-3-(6-methyl-7-oxo-6,7-dihydro-1H-pyrrolo[2,3-c]pyridin-4-yl)phenyl)ethanesulfonamide (ABBV-075); (2-cyclopropyl-5-(3,5-dimethyl-3H-113-isoxazol-4-yl)-1H-benzo[d]imidazol-7-yl)di (pyridin-2-yl) methanol (GS-5829, Alobresib); (S)-4-(6-(3,5-dimethylisoxazol-4-yl)-1-(1-(pyridin-2-yl)ethyl)-1H-pyrrolo[3,2-b]pyridin-3-yl)benzoic acid (PLX51107); methyl (S)-2-(4-(4-chlorophenyl)-2,3,9-trimethyl-6H-thieno[3,2-f][1,2,4]triazolo[4,3-a][1,4]diazepin-6-yl) acetate (MS417); TEN-010; ZEN003694; GSK2820151; FT-1101; olinone; compounds that interfere with binding of bromodomain-containing BET proteins to post-translationally modified histones (e.g., acetylated histones); compounds that disrupt formation of chromatin complexes critical for mRNA transcription, elongation, and splicing; compounds that compete with acetylated peptide binding (e.g., acetyl-lysine); ompounds that suppress Cytosine-phosphate-Guanine (CpG) island negative genes (CGI-genes) (e.g., ABBV-075; ABBV-744; 2-((S)-4-(4-chlorophenyl)-2,3,9-trimethyl-6H-thieno[3,2-f][1,2,4]triazolo [4,3-a][1,4]diazepin-6-yl)-N-(4-(2-(2-(2-(2-((2-(2,6-dioxopiperidin-3-yl)-1,3-dioxoisoindolin-4-yl) amino)ethoxy)ethoxy)ethoxy) ethoxy)phenyl)acetamide (ARV-825); AZD 5153; 2-[[[4-(1,2-Dihydro-2-methyl-l-oxo-2,7-naphthyridin-4-yl)-2-6-dimethoxyphenyl]methyl] methylamino]-N-[2-[2-[2-[[2-(2,6-dioxo-3-piperidinyl)-2,3-dihydro-1,3-dioxo-1H-isoindol-4-yl] amino]ethoxy]ethoxy]ethyl] acetamide dihydrochloride (dBRD9); MS645; PLX51107; CPI-203; I-BET 151; I-BET 762; MS417; OTX-015); and the like, or pharmaceutically acceptable enantiomers, diastereomers, racemates, and salts thereof.

In some embodiments, a BET inhibitor of the disclosure may be structurally similar to JQ1 and similarly binds to BRD4 at various positions, including but not limited to, tryptophan at position 81 (Trp-81), proline at position 82 (Pro-82), tyrosine at position 97 (Tyr-97), asparagine at position 140 (Asn-140), and methionine at position 149 (Met-149), while having limited binding to, but not limited to, leucine at position 94 (Leu-94), tyrosine at position 139 (Tyr-139), aspartic acid at position 144 (Asp-144), aspartic acid at position 145 (Asp-145), and isoleucine at position 146 (Ile-146), which is described by Jung et al. (JBC 289(13):9304-9319, 2014) and incorporated by reference in its entirety. Some embodiments may be directed to a BET inhibitor structurally similar to JQ1 that binds mutated BRD4, where leucine at position 94 and aspartic acid at position 145 are each modified to alanine. See, Jung et al. for characterization of exemplary BET inhibitors. For example, the BET inhibitor may be tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof. In some embodiments, the BET inhibitor candidate substance may be tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof.

Other embodiments of the invention provide for methods using a BET inhibitor or composition containing such a BET inhibitor and a pharmaceutically acceptable carrier thereof, to treat a subject suffering from an age-related inflammatory disease. In some embodiments, the BET inhibitor may be administered in a therapeutically effective amount of tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof, to the subject in need thereof, or a pharmaceutical composition containing tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof, to the subject in need thereof, and a pharmaceutically acceptable carrier. The methods of treating result in delaying aging, delaying the onset of the age-related inflammatory disease, and/or extending lifespan of the subject suffering from an age-related inflammatory disease or cells, such as senescent cells. Non-limiting examples of age-related inflammatory disease may include Alzheimer’s disease, Parkinson’s, chronic inflammation, Rheumatoid arthritis, maculopathy atherosclerosis, diabetes, stroke, myocardial infarction, heart failure, hypertension, osteoarthritis, osteoporosis, sarcopenia, loss of bone marrow, idiopathic pulmonary fibrosis, degraded immune function, age-related macular degeneration, abnormal proliferative diseases, and disorders associated with a decrease in hormones (e.g., testosterone, estrogen, growth hormone, insulin-like growth factor I (IGF-I)) or reduced energy production.

In some embodiments, the treatment may reduce or ameliorate the age-related inflammatory disease in a subject or in senescent cells and/or symptoms associated with the age-related inflammatory disease. Further embodiments of the methods of treating disclosed here result in delaying aging or the onset of age-related inflammatory disease, or extending lifespan of a subject suffering from an age-related inflammatory disease or cells, such as senescent cells, may be by 1% or more (e.g., 2%, 4%, 6%, 8%, 10%, 20%, 30%, 40%, 50% 60%, 70%, 80%), by 90% or less (e.g., 89%, 87%, 85%, 75%, 65%, 55%, 45%, 35%, 25%, 15%, 5%), or by 1% - 90% (e.g., 5% - 80%; 10% - 70%; 15% - 60%, 20% - 50%, 30% - 40%; 1% - 50%). The methods described here may treat a subject that is a mammal, such as but not limited to, a human, murine, canine, feline, or non-human primate.

Other embodiments may be directed to the administering step of a BET inhibitor or a pharmaceutical compositions containing the BET inhibitor and a pharmaceutically acceptable carrier in an effective amount, where the route of administration may be selected from, but not limited to, oral (e.g., tablets, capsules, caplets, cachets, lozenges, cachets, dragées, solutions, suspensions, gels, syrups; mucosal (sub- or supralingual or buccal (e.g., lozenges, pastilles), eye or ear drops, intranasal); parenteral injection (e.g., subcutaneous, intraperitoneal, intravenous, intradermal, intramuscular); topical (e.g., aqueous solution, suspension, ointment, cream, gel); inhalation (e.g., gas, aerosol, powder); transdermal (e.g., single-layer drug-in-adhesive, multi-layer drug-in-adhesive, reservoir, matrix, vapor patch, microneedles).

The BET inhibitors of the invention may be administered in an effective amount sufficient to provide a desired outcome as determined by a person of skill in the art using routine methods. In some embodiments, an effective amount is an amount that shows any improvement or lack of decline or worsening in the condition being treated. In various embodiments, the type and extent of the disease or condition being treated may dictate the effective amount and/or the need or the use of one or more additional therapeutic agents. Yet a person of ordinary skill in the art can determine suitable doses and ranges of therapeutic agents to use, for example, based on in vitro and/or in vivo testing and/or other knowledge of compound dosages.

An effective amount of BET inhibitor as described here typically will vary from 0.001 mg/kg or greater (e.g., 0.01 mg/kg, 0.1 mg/kg, 1 mg/kg, 10 mg/kg, 20 mg/kg, 40 mg/kg, 60 mg/kg, 80 mg/kg, 100 mg/kg, 200 mg/kg, 500 mg/kg, 1000 mg/kg); from 1000 mg/kg or less (e.g., 750 mg/kg, 500 mg/kg, 250 mg/kg, 150 mg/kg, 50 mg/kg, 25 mg/kg, 15 mg/kg, 5 mg/kg, 0.25 mg/kg, 0.15 mg/kg, 0.05 mg/kg, 0.025 mg/kg, 0.005 mg/kg, 0.002 mg/kg); from 0.001 mg/kg to 1000 mg/kg (e.g., 0.01 mg/kg to 750 mg/kg, 0.1 mg/kg to 500 mg/kg, 1.0 mg/kg to 250 mg/kg, 10.0 mg/kg to 150 mg/kg) in one or more dose administrations, for one or several days, consecutively or intermittently (depending on the mode of administration and the factors discussed here).

In some embodiments, the BET inhibitors of the disclosure may be formulated in an oral composition using hydroxypropyl methylcellulose phthalate (HPMCP) or thiolated HPMCP (T-HPMCP), which may be synthesized, for example, by modifying HPMCP as described by Singh et al. (Biomaterials 59:144-159, 2015). Oral delivery of the BET inhibitors may be by, for example, microparticles prepared as described in Singh et al. (incorporated here by reference in its entirety for all of its teachings), where the diameter of the particles may be 0.01 µm -20 µm (e.g., 0.05 µm - 18 µm; 0.1 µm - 16 µm; 0.5 µm - 14 µm; 1 µm - 12 µm; 2 µm - 10 µm; 3 µm - 8 µm; 4 µm - 6 µm); 0.01 µm or greater (e.g., 0.03 µm; 0.07 µm; 0.09 µm; 0.3 µm; 0.7 µm; 0.9 µm; 3 µm; 5 µm; 7 µm; 9 µm; 11 µm; 13 µm; 15 µm; 17 µm; 19 µm); or 20 µm or less (e.g., 18 µm; 16 µm; 14 µm; 12 µm; 10 µm; 8 µm; 6 µm; 4 µm; 2 µm; 1.8 µm; 1.6 µm; 1.4 µm; 1.2 µm; 0.8 µm; 0.6 µm; 0.4 µm; 0.2 µm; 0.08 µm; 0.06 µm; 0.04 µm; 0.02 µm).

Some embodiments of the invention may provide a method of inhibiting CGI- gene misexpression in a cell by administering an effective amount of a BET inhibitor to the cell. The cell may be a mammalian cell (e.g., human, murine, canine, feline), where the mammalian cell may be one or more cells selected or derived from, but not limited to, an aging cell, a senescent cell, a progeria cell, a pancreatic cell (e.g., pancreatic β-cell), a fibroblast cell (e.g., tail-tip fibroblast cell), an embryonic stem cell (e.g., a Hp1α-Knockdown cell), an epithelial cell (e.g., murine lung epithelial cell, a Lmnb1-Knockdown cell), a myoblast cell (e.g., Lbr^{-/-} cell), a kidney cell, a heart cell, a muscle cell, a neuronal cell, a pulmonary cell, or a bone cell.

In another embodiment, the method of inhibiting CGI- gene misexpression in a cell by administering an effective amount of a BET inhibitor or candidate substance that acts or functions as a BET inhibitor, where the cell may be a mammalian cell (e.g., human, murine, canine, feline), a senescent cell, or a cell present in an age-related inflammatory disease (e.g., Alzheimer’s disease, Parkinson’s, chronic inflammation, Rheumatoid arthritis, maculopathy atherosclerosis, diabetes, stroke, myocardial infarction, heart failure, hypertension, osteoarthritis, osteoporosis, sarcopenia, loss of bone marrow, idiopathic pulmonary fibrosis, degraded immune function, age-related macular degeneration, abnormal proliferative diseases, and disorders associated with a decrease in hormones (e.g., testosterone, estrogen, growth hormone, insulin-like growth factor I (IGF-I)) or reduced energy production). The method of inhibiting CGI- gene misexpression in such a cell may be by administering an effective amount of a BET inhibitor to the cell resulting in one or more of inhibition of nuclear lamina and/or heterochromatin disruption, inhibition of CGI- misexpression associated organ failure, or inhibition of age-associated degenerative changes (e.g., neurodegeneration, memory loss, osteoporosis, macular degeneration, hearing loss, heart disease, vascular disease, diabetes, metabolic syndrome, decreased organ function (e.g., lung, kidney), sarcopenia, frailty).

One embodiment of the invention may provide a CGI gene expression assay for identifying the modification of CGI- gene expression in control (e.g., young) versus aging or senescent cells. This system provides a tool to study senescent mammalian cells and different forms of senescence-directed interventions using BET inhibitor candidate substances in an in vitro model, which may be ideal for high-throughput and pipeline acceleration. Approaches that allow a high-throughput and cell focused exploration of senescence-related biological processes are desired. For example, screening various BET inhibitor candidate substances and their effects on gene expression in control (i.e., young, healthy) cells as compared to aging or senescent cells. The cells may be mammalian cells (e.g., human, murine, canine, feline). Upregulation of many genes, including CGI- genes without candidate substances may be decreased with a BET inhibitor candidate substance.

Another embodiment provides for a method of screening potential candidate substances, such as, but not limited to, BET inhibitor candidate substances using any of the model systems described here. In one embodiment, the candidate substance may be any that reverses senescent cells to non-senescent cells, prevents the progression of non-senescent cells to senescent cells, induces senescent cell death, suppresses senescent phenotypes without cell killing, induces toxicity to proliferating cells, induces toxicity to senescent cells, suppresses cell senescence, increases in cell senescence, or the like. The screening method described here may be a high-throughput, low cost method of identifying potential candidate substances that may be a BET inhibitor or act or function as a BET inhibitor. Any candidate substance found to delay or inhibit senescence through a BET inhibitor or substance that may act or function as a BET inhibitor may be useful in a treatment and/or prevention of an aging or age-related inflammatory disease.

In yet another embodiment, a method of treating a subject suffering from an aging or age-related disease or disorder may be personalized by screening candidate substances against the subject’s own cells, e.g., human primary cells from the subject that are transplanted into the model system described here. Quantifying a decrease in senescent cells or observing a decrease or reversal of senescent cells to non-senescent cells as described here with administration of the candidate substance, thereby provides a personalized approach to therapeutic medicine.

Any of the embodiments to methods described here may be in a high-throughput or automated format. In some embodiments, the method of screening described here may be a high-throughput format, providing a rapid and time- and cost-efficient process for analyzing cells for cellular senescence and identifying BET inhibitor candidate substances that delay senescence or aging, or extend lifespan.

The therapeutic methods of the invention (which include prophylactic treatment) in general comprise administration of a therapeutically effective amount of the candidate substance identified and described herein, to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like). The compounds herein may be also used in the treatment of any other disorders in which aging or age-related diseases or disorders, or in other embodiments, excessive or abnormal proliferation, may be implicated.

In one embodiment, the invention provides a method of monitoring treatment progress. The method includes determining a level of a diagnostic marker, such as one for cellular senescence (e.g., any target delineated herein modulated by a candidate substance described or identified by methods disclosed here, a protein or indicator thereof, etc.) or diagnostic measurement (e.g., screen, assay, quantification of cellular senescence) for identifying, for example, a reversal of senescent cells to non-senescent cells, prevention of progression of non-senescent cells to senescent cells, senescent cell death, suppression of senescent phenotypes without cell killing, toxicity to proliferating cells, toxicity to senescent cells, suppression of cell senescence, increase in cell senescence, or increase in proliferation, in a subject suffering from or susceptible to a disorder or symptoms thereof associated with aging, age-related disease or condition, or diseases or conditions of excessive or abnormal proliferation, in which the subject has been administered a therapeutic amount of a BET inhibitor or candidate substance that acts or functions as a BET inhibitor (e.g., tert-butyl (S)-2-(4-(4-chlorophenyl)-2,3,9-trimethyl-6H-thieno[3,2-f][1,2,4]triazolo[4,3-a][1,4]diazepin-6-yl)acetate; (R)-7-(3,5-dimethylisoxazol-4-yl)-8-methoxy-1-(1-(pyridin-2-yl)ethyl)-1,3-dihydro-2H-imidazo[4,5-c]quinolin-2-one; (S)-2-(6-(4-chlorophenyl)-8-methoxy-1-methyl-4H-benzo[f][1,2,4]triazolo [4,3-a] [1,4]diazepin-4-yl)-N-ethylacetamide; (S)-2-(6-(4-chlorophenyl)-8-methoxy-1-methyl-4H-benzo [f][1,2,4]triazolo[4,3-a][1,4]diazepin-4-yl)-N-ethylacetamide; (S)-2-(4-(4-chlorophenyl)-2,3,9-tri methyl-6H-thieno[3,2-f][1,2,4]triazolo[4,3-a][1,4]diazepin-6-yl)acetamide; (S)-2-(6-(4-chlorophenyl) -1-methyl-4H-benzo[c]isoxazolo [4,5-e]azepin-4-yl)acetamide; 2-(4-(2-hydroxyethoxy)-3,5-dimethyl phenyl)-5,7-dimethoxyquinazolin-4(3H)-one; (Z)-4-(2-(2-amino-5-methyl-4-oxocyclohexa-2,5-dien-1-ylidene)hydrazineyl)-N-(pyridin-2-yl)benzenesulfonamide; 2-methoxy-N-(3-methyl-2-oxo-1,2,3,4-tetrahydroquinazolin-6-yl)benzenesulfonamide; N-ethyl-4-(2-(4-fluoro-2,6-dimethylphenoxy)-5-(2-hydroxypropan-2-yl)phenyl)-6-methyl-7-oxo-6,7-dihydro-1H-pyrrolo[2,3-c]pyridine-2-carboxamide; 2-Morpholin-4-yl-8-phenylchromen-4-one; (R)-4-(2-(4-(1-(3-methoxy-[1,2,4]triazolo [4,3-b] pyridazin-6-yl)piperidin-4-yl)phenoxy) ethyl)-1,3-dimethylpiperazin-2-one; N,N′-(3,6,9,12, 15, 18,21-heptaoxatricosane-1,23-diyl)bis(2-((S)-4-(4-chlorophenyl)-2,3,9-trimethyl-6H-thieno[3,2-f][1,2,4] triazolo[4,3-a][1,4] diazepin-6-yl) acetamide); (S)-N,N′-(decane-1,10-diyl)bis(2-((6S)-4-(4-chloro phenyl)-2,3,9-trimethyl-6H-thieno[3,2-f][1,2,4]triazolo[4,3-a][1,4] diazepin-6-yl)acetamide); 2-(4-(2-(isopropylamino)ethoxy)-3,5-dimethylphenyl)-5,7-dimethoxy quinazolin-4(3H)-one; (S)-7,8-di methoxy-N,4-dimethyl-1-(4-(4-methylpiperazin-1-yl)phenyl)-4,5-dihydro-3H-benzo[d][1,2] diazepine -3-carboxamide; (S)-6-(3,5-dimethylisoxazol-4-yl)-3-(pyridin-2-yl)-3,4-dihydro-5-oxa-1,2a-diaza acenaphthylen-2(1H)-one; 6-(3-hydroxypropyl)-2-(1,3,6-trimethyl-2-oxo-2,3-dihydro-1H-benzo [d]imidazol-5-yl)-1H-benzo[de]isoquinoline-1,3(2H)-dione;(S)-2-(3-(1,4-dimethyl-1H-1,2,3-triazol-5-yl)-5-(phenyl(tetrahydro-2H-pyran-4-yl)methyl)-5H-pyrido[3,2-b]indol-7-yl)propan-2-ol; N-(4-(2,4-difluorophenoxy)-3-(6-methyl-7-oxo-6,7-dihydro-1H-pyrrolo[2,3-c]pyridin-4-yl)phenyl)ethane sulfonamide; (2-cyclopropyl-5-(3,5-dimethyl-3H-113-isoxazol-4-yl)-1H-benzo[d]imidazol-7-yl)di (pyridin-2-yl) methanol; (S)-4-(6-(3,5-dimethylisoxazol-4-yl)-1-(1-(pyridin-2-yl)ethyl)-1H-pyrrolo [3,2-b]pyridin-3-yl)benzoic acid; methyl (S)-2-(4-(4-chlorophenyl)-2,3,9-trimethyl-6H-thieno[3,2-f][1,2,4]triazolo[4,3-a][1,4]diazepin-6-yl)acetate; TEN-010; ZEN003694; GSK2820151; FT-1101; olinone; compounds that interfere with binding of bromodomain-containing BET proteins to post-translationally modified histones (e.g., acetylated histones); compounds that disrupt formation of chromatin complexes critical for mRNA transcription, elongation, and splicing; compounds that compete with acetylated peptide binding (e.g., acetyl-lysine); compounds that suppress Cytosine-phosphate-Guanine (CpG) island negative genes (CGI- genes) (e.g., ABBV-075; ABBV-744; ARV-825; AZD 5153; 2-[[[4-(1,2-Dihydro-2-methyl-1-oxo-2,7-naphthyridin-4-yl)-2-6-dimethoxy phenyl]methyl]methylamino]-N-[2-[2-[2-[[2-(2,6-dioxo-3-piperidinyl)-2,3-dihydro-1,3-dioxo-1H-isoindol-4-yl]amino]ethoxy]ethoxy]ethyl] acetamide dihydrochloride; MS645; PLX51107; CPI-203; I-BET 151; I-BET 762; MS417; OTX-015); and the like, or pharmaceutically acceptable enantiomers, diastereomers, racemates, and salts thereof) described herein sufficient to treat the disease or symptoms thereof. The level of cellular senescence determined in the method can be compared to control in either healthy, young normal controls or in other afflicted subjects to establish the subject’s disease status. In some embodiments, a second level of marker in the subject may be determined at a time point later than the determination of the first level, and the two levels may be compared to monitor the course of disease or the efficacy of the therapy. In certain embodiments, a pre-treatment level of a marker in the subject is determined prior to beginning treatment according to this invention; this pre-treatment level of a marker can then be compared to the level of the marker in the subject after the treatment commences, to determine the efficacy of the treatment.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what may be regarded as the invention.

### EXAMPLES

The following examples illustrate specific aspects of the instant description. The examples should not be construed as limiting, as the example merely provides specific understanding and practice of the embodiments and its various aspects.

### Example 1: CpG Island-Mediated Chromatin Architecture

Studies identified new functions of CpG islands (CGIs) with respect to the molecular mechanisms underlying aging. CGIs are DNA elements with high frequencies of Cytosine-phosphate-Guanine (CpG) dinucleotides and are typically associated with gene promoters. In mammals, about 60% of all genes contain CGIs (CGI positive (CGI+) genes) while the other 40% do not (CGI negative (CGI-) genes). CGI+ genes are broadly expressed throughout the body (such as, housekeeping genes, e.g., α-tubulin, β-actin, GAPDH) and developmentally regulated genes (e.g., SOX2, HOX gene clusters, MYOD1). CGI- genes exhibit more tissue-restricted expression patterns, which include, but are not limited to, Myosin heavy/light-chain genes, Casein genes, Olfactory receptor genes; genes involved in innate immune response (e.g., Tumor Necrosis Factor α, Interleukins (e.g., IL-1β, IL-6, IL-8, IL-10); CC and CXC chemokines.

Computational analysis demonstrated that CGI+ and CGI- genes are regulated by completely distinct mechanisms (FIGS. 1C-1E). In eukaryotes, inactivation of gene expression occurred primarily by interaction with Polycomb group proteins or the formation of heterochromatin. As shown in FIG. 1C and FIG. 1D, Polycomb proteins were found to preferentially occupy inactive CGI+ genes, and rarely occupied either inactive or active CGI- genes. Polycomb group (PcG) proteins are epigenetic regulators of transcription that play an important role in stem cell identity, differentiation, and disease. They function within Polycomb repressive complexes (PRCs) (i.e., multi-protein complexes), which modify histones, among other proteins, and silence target genes. The signal of trimethylated lysine 9 of histone H3 (H3K9me3), a heterochromatin mark, was high in inactive CGI- genes, but not in CGI+ genes. CGI+ genes sustained characteristics of euchromatin (i.e., DNA demethylation (DNA-me) and high levels of chromatin accessibility), even when they are transcriptionally inactive. Taken together, these data showed that CGI- genes were silenced by heterochromatin formation, while CGI+ genes remain as euchromatin, even when repressed by Polycomb proteins (FIG. 1D). CGI+ and CGI- genes exhibited distinct nuclear localization patterns under normal conditions, as shown in a revised model (FIG. 1E); only CGI- genes, but not CGI+ genes, associated with the nuclear lamina when transcriptionally inactive.

Aged nuclei exhibited global decondensation of heterochromatin, and degradation of nuclear lamina components (FIG. 1B). Data showed that lamina association and heterochromatin formation were exclusive characteristics of inactive CGI- genes (FIGS. 1C-1E). Heterochromatin decondensation and nuclear lamina disruption during aging specifically hindered the repression of CGI- gene expression, resulting in their uncontrolled expression (FIG. 1F).

### Example 2: Heterochromatin, the Nuclear Lamina, and CPG Islands Link During Aging

Although CGI elements were discovered over three decades ago, their critical influence on gene regulation has long been overlooked. Previous results revealed their importance to gene regulation and chromatin architecture, demonstrating that only CGI- genes can form heterochromatin and associate with the nuclear lamina.

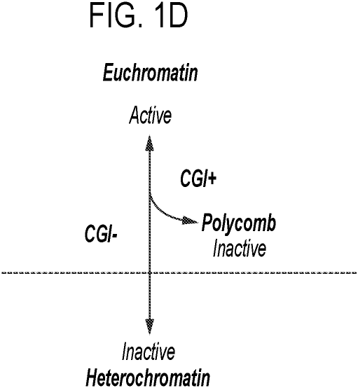

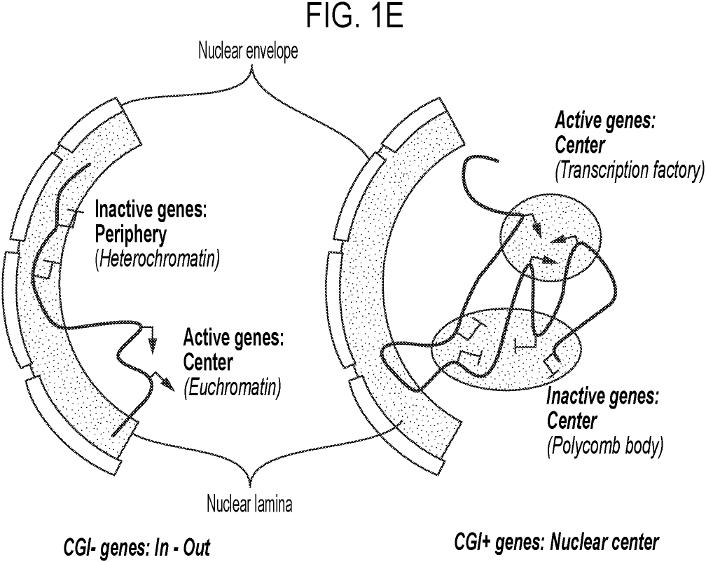

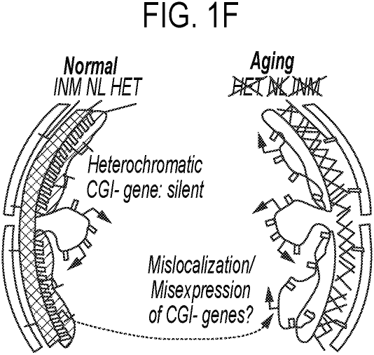

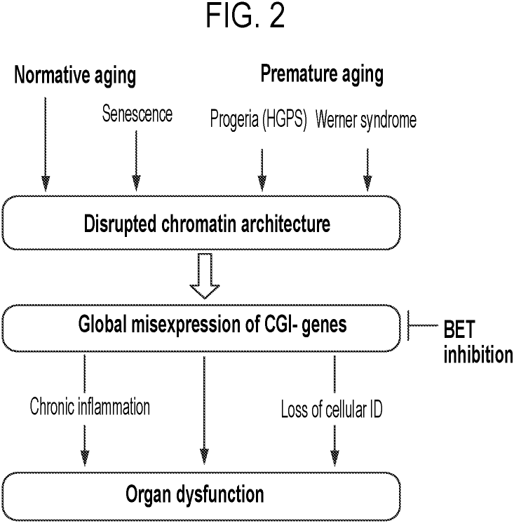

Changes in chromatin architecture during aging have been recognized for more than two decades. However, the mechanisms by which these structural changes contribute to age-related pathologies were not clear. Aging-mediated deterioration results from the disrupted regulation of CGI- genes are shown in FIG. 2. Understanding how changes in chromatin architecture give rise to aging-related defects yield new insights into the pathophysiology of aging, as well as a new and effective therapeutic strategy against aging and age-related diseases.

Preliminary results demonstrated that changes in nuclear chromatin architecture are a hallmark of aging; aged nuclei exhibited disruption of the nuclear lamina and decondensation of associated heterochromatin. Genes associated with CpG islands (CGI+ genes) were broadly expressed throughout the body, while genes lacking CGIs (CGI- genes) generally remained silent and are expressed in tissue-restricted manners. In young nuclei, among all silent genes, only CGI- genes formed heterochromatin and associated with nuclear lamina.

### Example 3: Global Misexpression of CGI- Genes in Aged Mouse Kidneys

Age-mediated gene expression changes in mouse tissues were characterized in vivo. Specifically, kidney gene expression data generated from Diversity Outbred (“DO”) mice (provided by the Nathan Shock Center of Excellence at The Jackson Laboratory, Bar Harbor, ME) were analyzed. DO mice are a genetically diverse mouse resource that mimics the complexity of the human population with variable rates of aging.

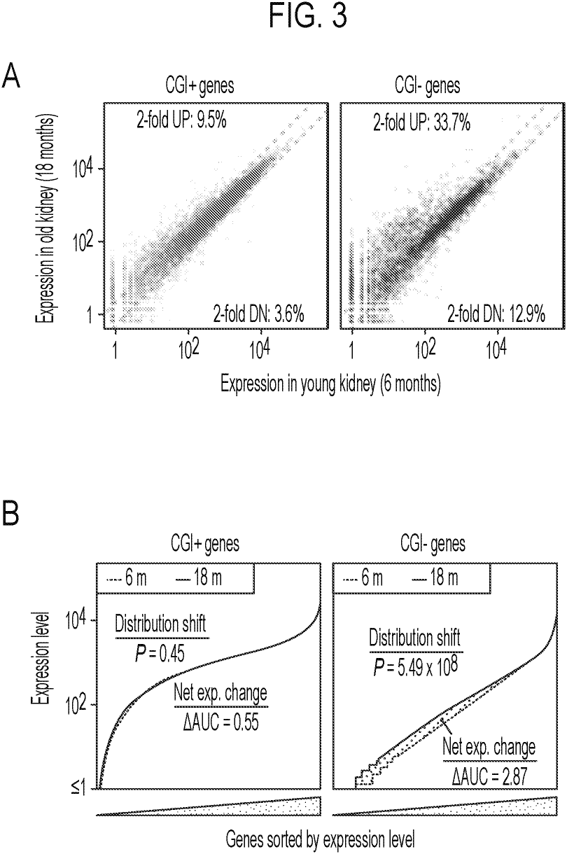

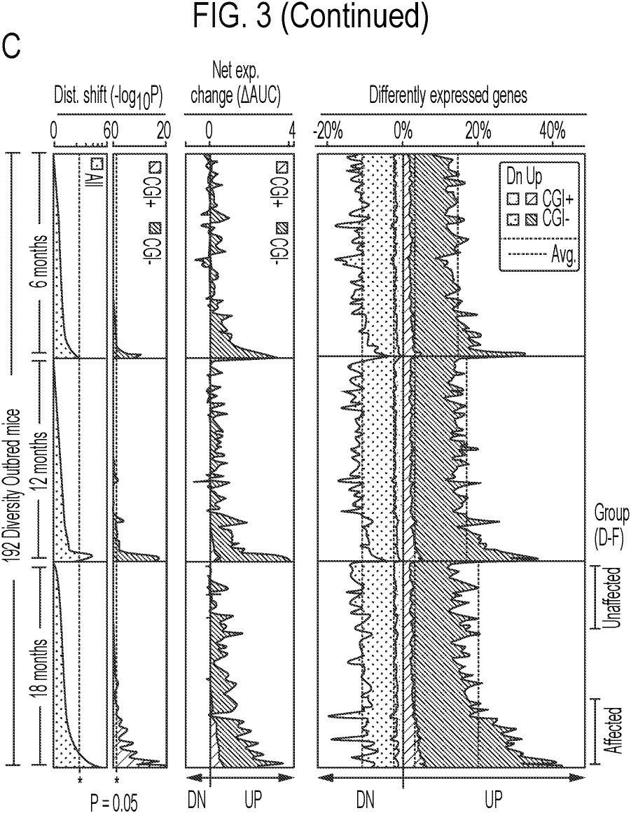

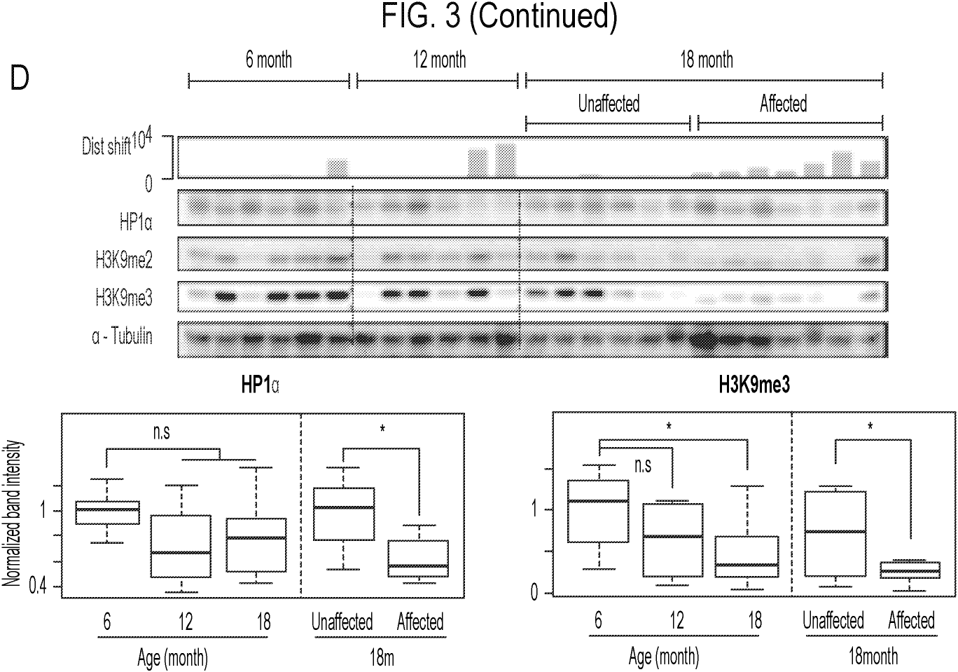

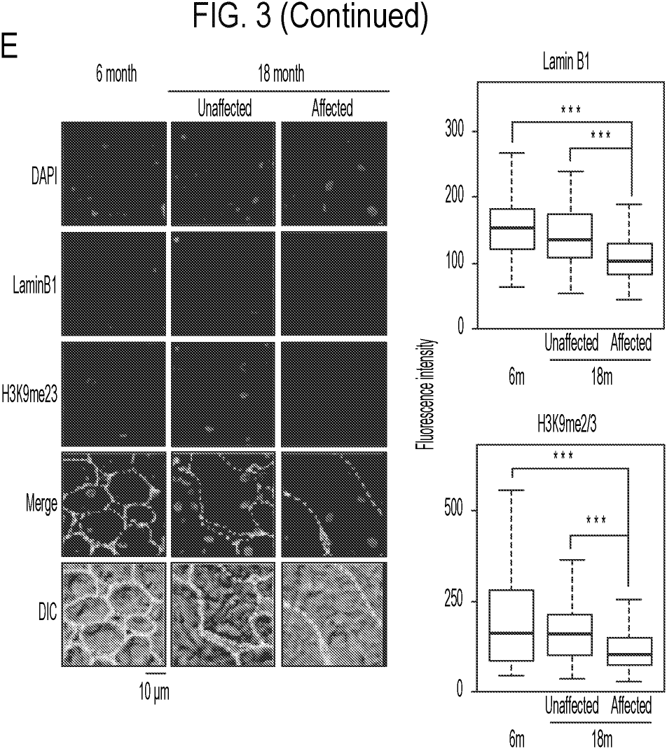

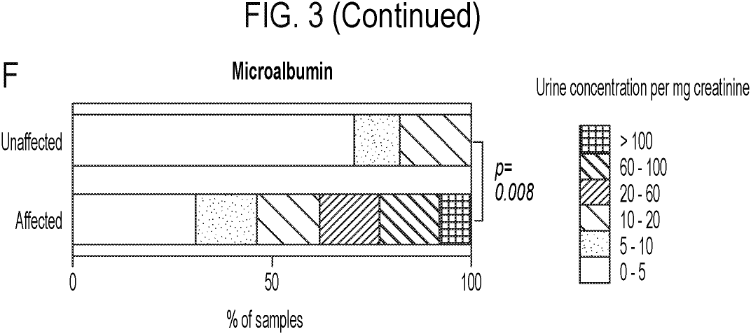

FIG. 3A shows an example of gene expression in kidneys from 6- and 18-month-old DO mice, where over 30% of all CGI- genes were globally upregulated over 2-fold in the aged kidney. To systematically monitor this pattern, two values representing the significance (distribution shift) and the directionality (net expression change) of the gene expression changes in each sample compared to young healthy kidneys were measured (FIG. 3B). FIG. 3C shows 192 DO mice from 3 age groups. Nearly all aged mice showing significant expression changes exhibited the same directionality; i.e., upregulation of many genes, especially CGI- genes. Roughly, up to 40% of CGI- genes were upregulated over 2-fold in the old kidneys. These patterns were more frequently observed in aged than young mice (vertical red dotted lines under differentially expressed genes, p = 9.2x10⁻⁹). These data support our hypothesis that normative aging causes global misexpression of CGI- genes in vivo.

Not all aged kidneys show misexpression of CGI- genes. To understand the nature of these differences, comparative analyses of aged kidneys with and without CGI- gene misexpression were performed (FIG. 3C, right; affected and unaffected, hereafter). As shown in a western blot analysis (FIG. 3D), most affected kidneys showed evident loss of heterochromatin marks (H3K9me⅔) and components (HP1α) compared to unaffected kidneys. Immunofluorescence analysis (FIG. 3E) showed a dramatic loss of nuclear lamina components (lamin B1) and heterochromatin marks (H3K9me⅔) in affected kidneys, compared to unaffected kidneys, implying that disorganization of nuclear/chromatin architecture is directly associated with CGI- gene misexpression during aging. These results showed that loss of heterochromatin marks is greater in aged than in young kidneys (FIG. 3D, FIG. 3E) demonstrating that heterochromatin decondensation during aging, previously shown only in cell cultures, also took place during normative mammalian tissue aging. Affected kidneys with CGI- gene misexpression showed a higher incidence of renal dysfunction, as evidenced by albuminuria (i.e., albumin in urine) (FIG. 3F). This indicated that CGI- gene misexpression is directly associated with deterioration of organ function during aging.

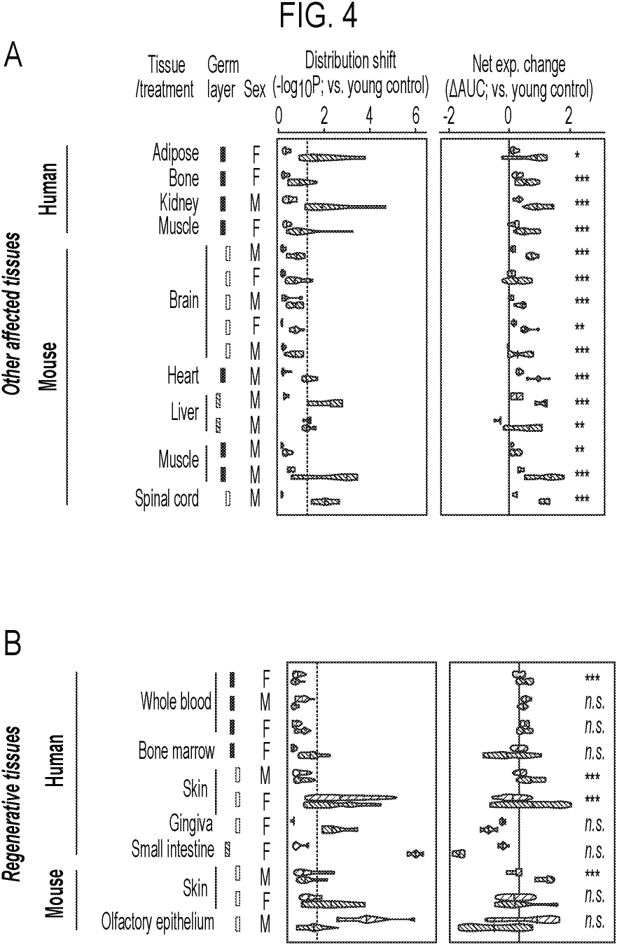

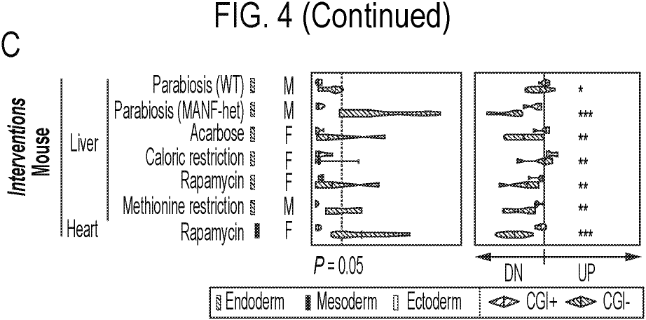

To test whether the misexpression of CGI- genes was also observed in other aged tissues, a systematic meta-analysis of published RNA-seq data was performed. For this, high-quality RNA-seq data (minimum reads ≥ 5x10⁶; n ≥ 5 in each group) generated from post-puberty young (18-30y in human, 1-6 m in mice) and old (≥50 y in human, ≥12 m in mice) tissues, and the significance (distribution shift) and the directionality (net exp. change) of gene expression changes during aging was monitored. As shown in FIG. 4A, multiple aged tissues show significant misexpression of CGI-genes compared to young tissues. Interestingly, highly regenerative tissues (e.g., blood and epithelial lineages) showed very little misexpression of CGI- genes (FIG. 4B). Together, these data indicated that CGI- gene misexpression was common in a wide range of aged tissues, and that this pattern was more evident in post-mitotic cells with accumulated cellular stress than in regenerative tissues.

FIG. 4C showed that various previously validated anti-aging strategies, such as parabiosis, caloric/methionine restriction, and rapamycin/acarbose treatment, could effectively revert/suppress CGI- gene misexpression. Taken together, these data demonstrated that CGI- gene misexpression is commonly observed in various aged tissues, providing a novel biomarker of aging.

### Example 4: Experimental Data

Scientific rigor and reproducibility may be ensured by the use of appropriate statistical tests, sufficient sample sizes, and an experimental/analytical design employing proper controls. All experiments and subsequent data analyses are performed in blinded and randomized fashions. The methods for statistical testing of results are determined by the type of experimental variables. For example, for DNA-fluorescence in situ hybridization (FISH) experiments, the data may be categorical in nature (e.g., nuclear periphery vs. center), and may not follow normal distributions. These data may be analyzed with nonparametric tests. In other experimental data analyses, statistical tests based on parametric distributions may be used, whenever applicable. All mouse experiments may be performed with the same number of males and females. The number of mice in each experiment may be determined using a power calculation (α = 0.05, minimum power = 80%), considering the types of variables (i.e., dichotomous or continuous) and study group design (e.g., two independent groups or one group versus population). Controls for each experiment may be selected by considering diverse variables, including genetic background, disease state, source of cells/tissues, gender, and age of donors.

### Example 5: Aging May Cause Mislocalization of CGI- Genes

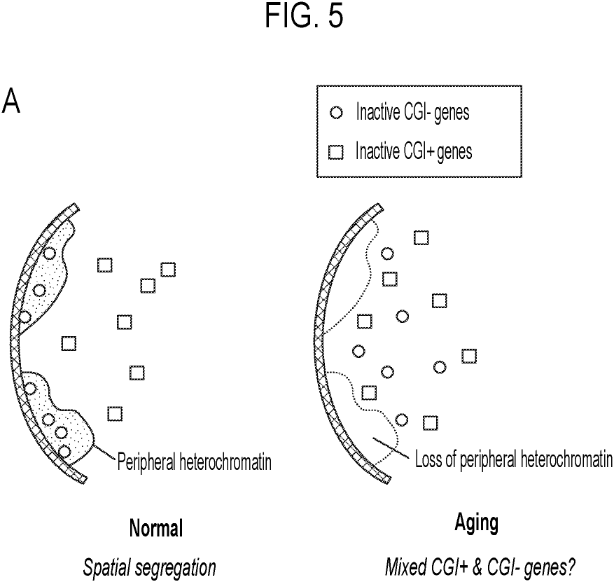

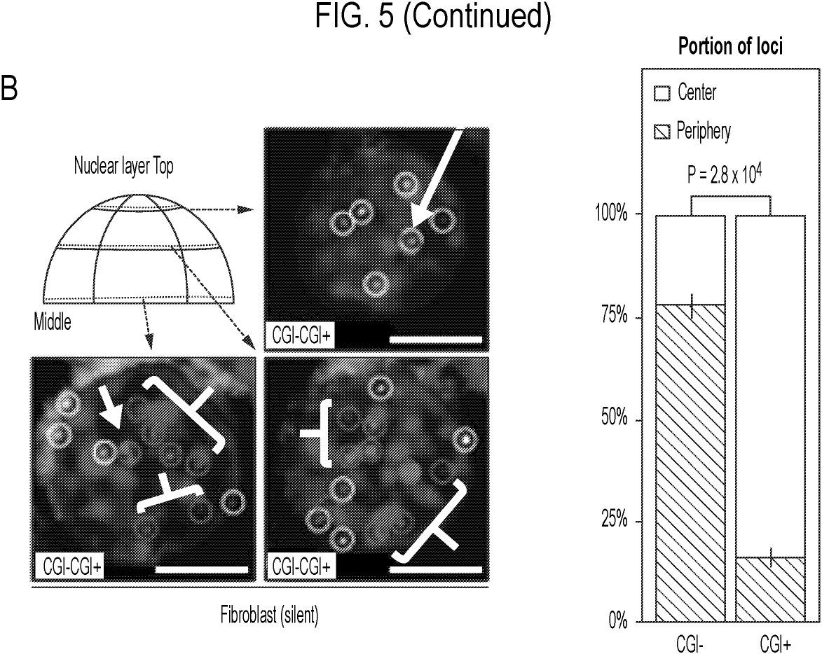

FIG. 5A illustrates that CGI- genes may lose the normal localization patterns in aged nuclei because of nuclear lamina and heterochromatin disruption in these nuclei. FIG. 5B shows CGI- gene localization patterns characterized using a multi-loci 3-D FISH (fluorescence in situ hybridization) assay (Beck et al. Nucleic Acids Res. 46:4382-4391, 2018) allowing simultaneous detection of ~10 genomic loci in a single nucleus. This technique may also be applied to paraffin-embedded or frozen tissue sections.

### Example 6: Nuclear Architecture Disruption Causes CGI- Gene Misexpression

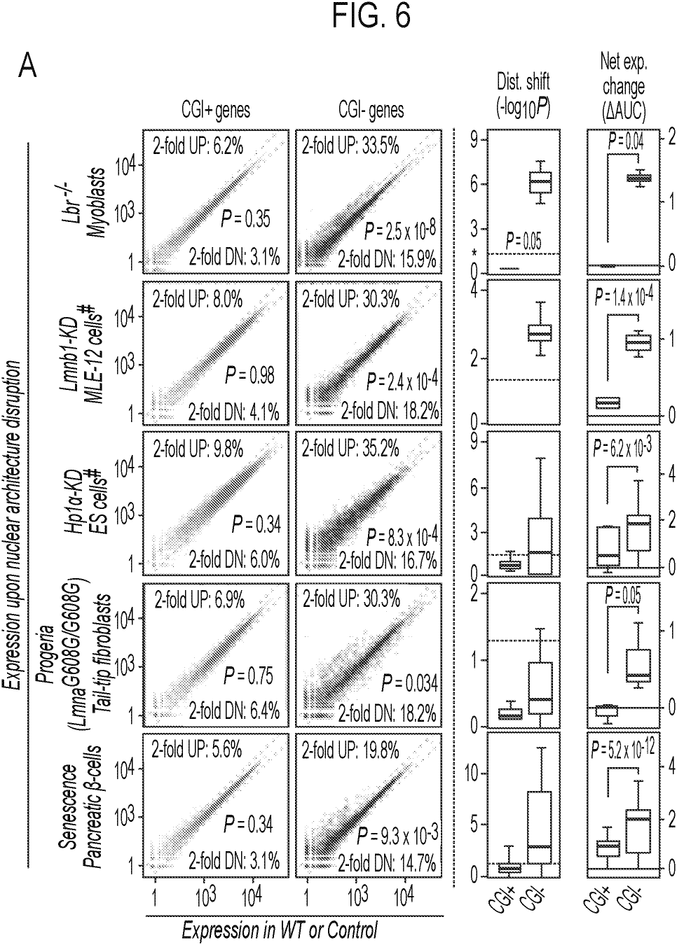

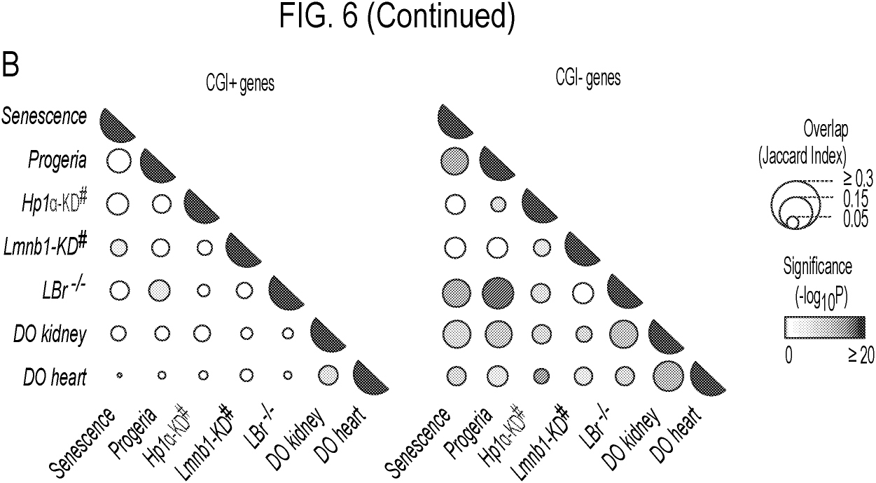

Depletion of the nuclear lamina (Lbr: lamin B receptor, Lmnb1: lamin B1) or of heterochromatin (HP1α) components that were shown to be degraded during aging (FIGS. 1A-1B) directly triggered misexpression of CGI- genes (FIG. 6A). Other aging contexts with disrupted nuclear/chromatin architectures [(i.e., progeria (HGPS: Hutchinson-Gilford progeria syndrome) and cellular senescence (pancreatic β-cells)] also exhibited misexpression of CGI- genes. As shown in FIG. 6B, CGI- genes upregulated upon nuclear lamina/heterochromatin disruption were also misexpressed during normative aging (DO kidney and heart) or in progeria/cellular senescence.

### Example 7: CGI- Gene Misexpression Drives Age-Associated Degenerative Changes

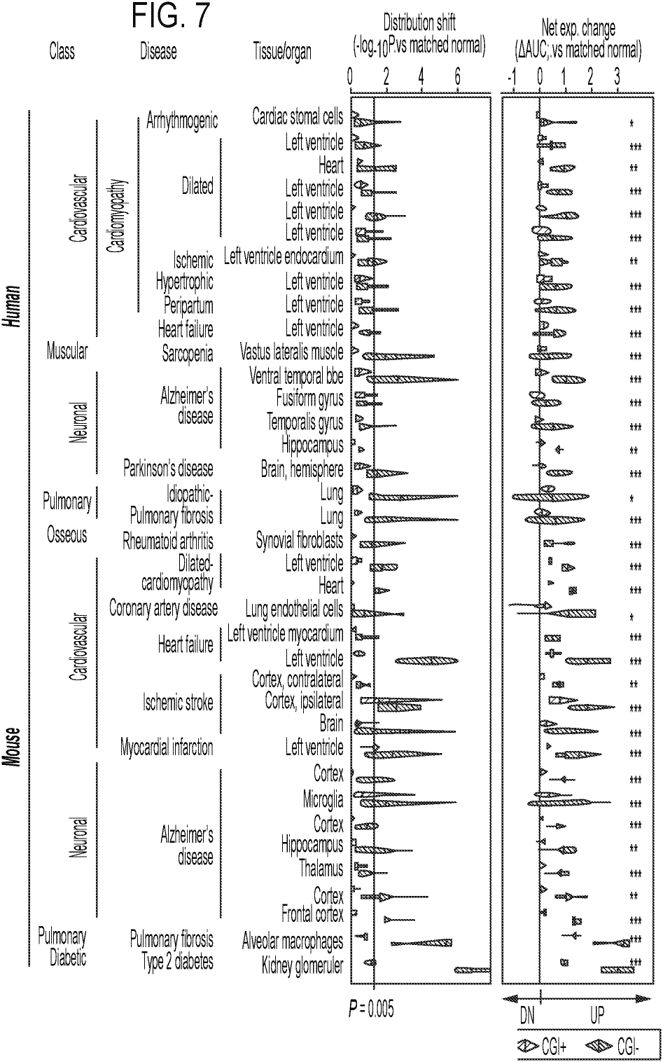

FIG. 7 shows that in various age-related diseases, misexpression of CGI- genes occurred when compared to normal tissues. It was found that the misexpression of CGI- genes is a driver of age-associated degenerative changes.

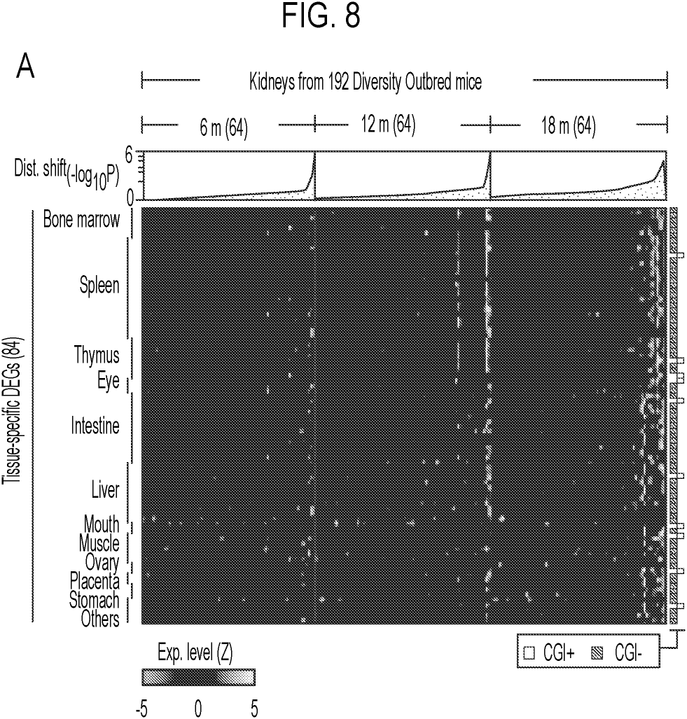

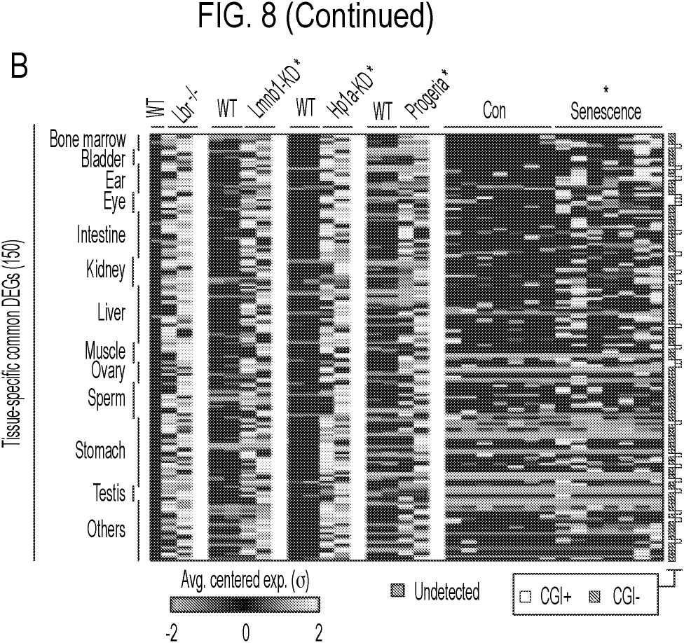

FIG. 8A shows that numerous tissue-specific genes expressed in non-kidney organs, such as the spleen, thymus, adipose tissue, eye, intestine, liver, etc, were expressed in old affected kidneys. The majority of these (88.1%) were CGI- genes. This pattern was also clearly recapitulated upon nuclear lamina/ heterochromatin disruption, as well as in progeria/cellular senescence (FIG. 8B). These results suggested that age-mediated chromatin architecture disruption, and subsequent misexpression of CGI- genes, is a driver for the loss of cellular identity.

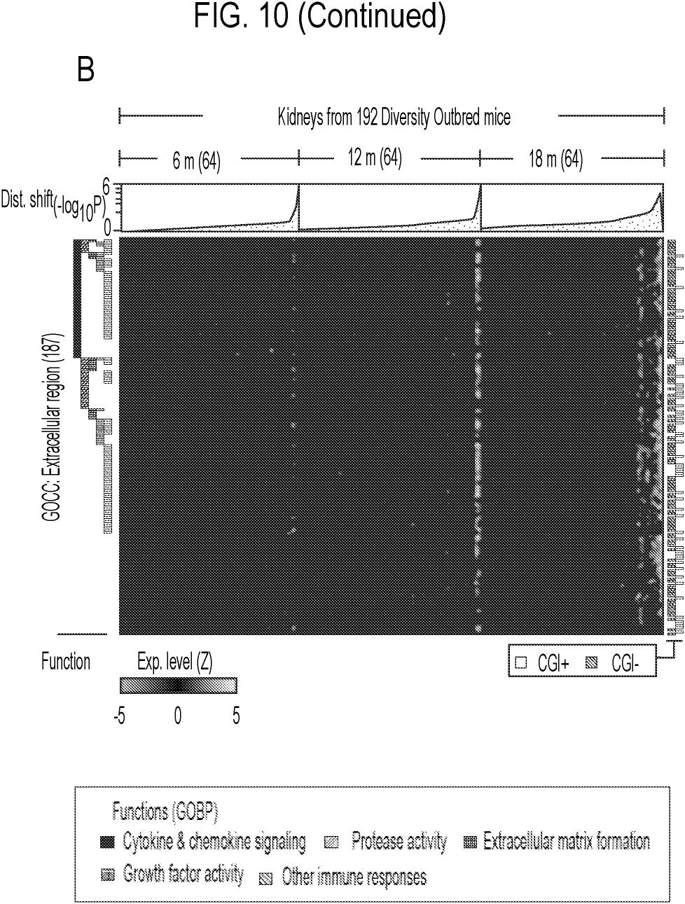

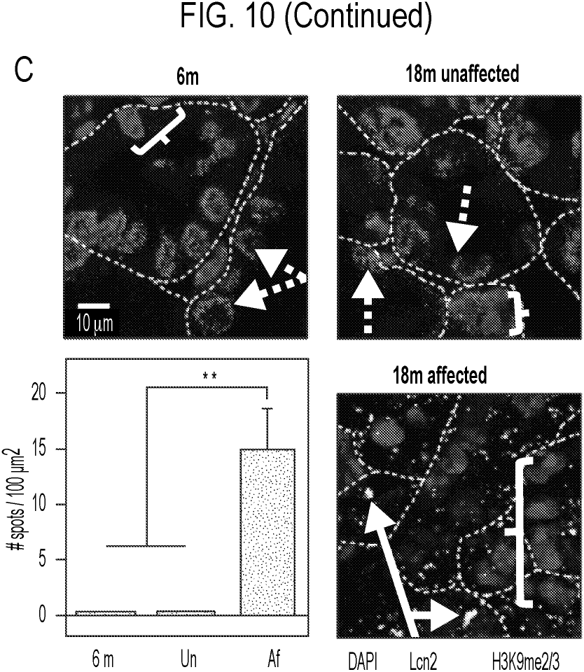

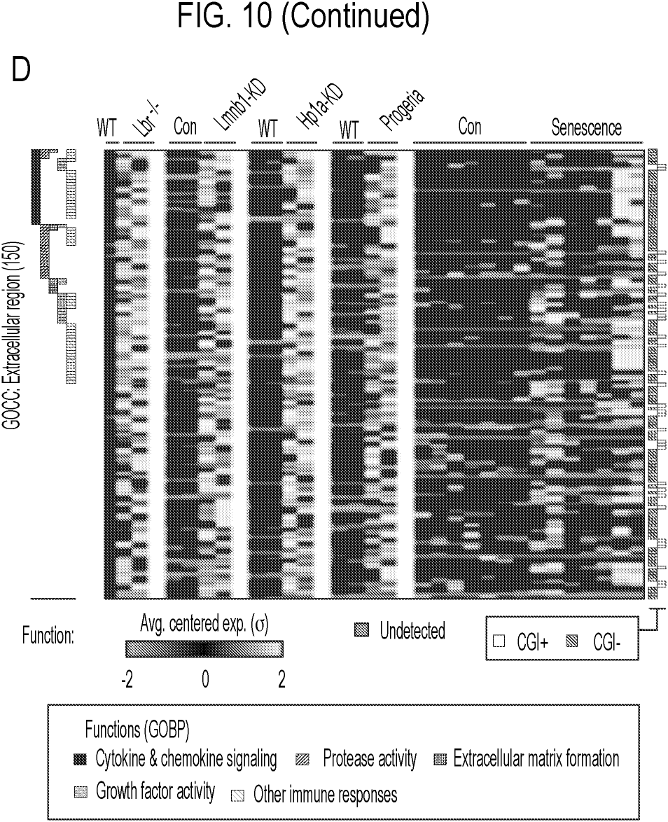

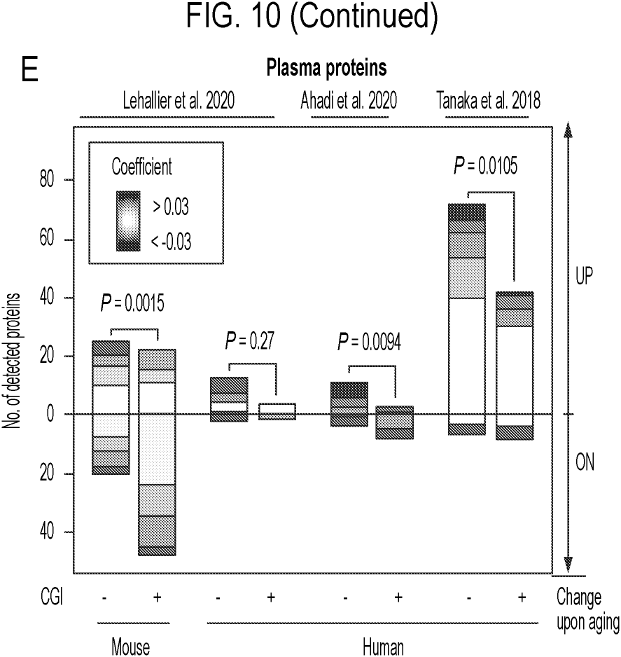

The misexpression of tissue-specific genes during aging (FIG. 8) was primarily from transcriptome analysis, which may be verified by tissue-staining using affected aged kidney samples. By performing mRNA-FISH (RNAscope®) on affected DO kidneys, organ-specific CGI- genes that are not normally expressed in healthy young kidneys, including Csn3 (_{κ}-casein; mammary gland-specific), Krt20 (keratin 20; intestine-specific), and Gpnmb (glycoprotein nonmetastatic melanoma protein B; expressed in nerves, bladder, genital pat pad) may be identified as misexpressed in the kidneys of affected DO mice. Aged but unaffected kidneys, as well as young kidneys may be used as negative controls. RNAscope signals may be quantified as the number of signal spots per 100 µm² (for example, see FIG. 10C).

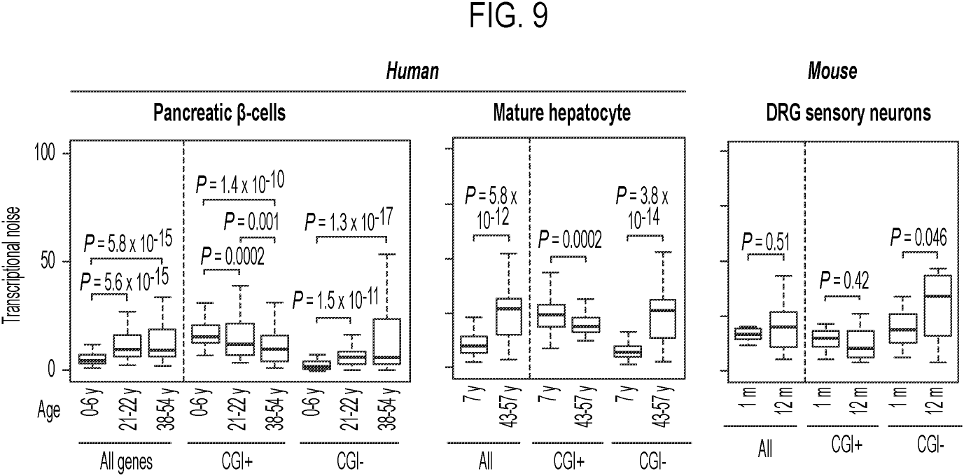

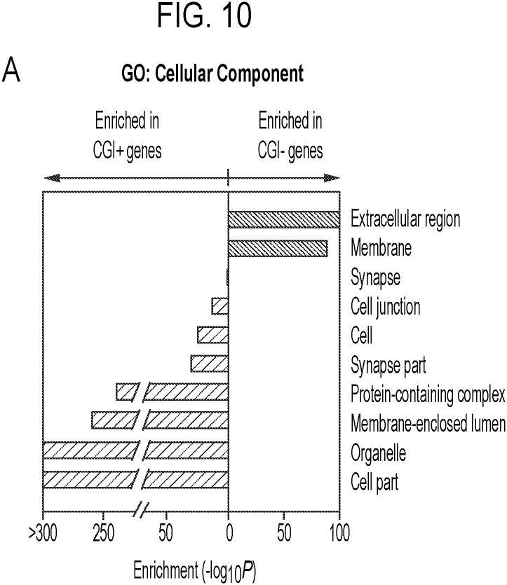

Based on single-cell RNA-seq (scRNA-seq) experiments, aging has been shown to increase cell-to-cell transcriptional variability (“transcriptional noise”) that, in turn, lead to loss of cellular identity and impaired tissue homeostasis (see, e.g., Swisa et al. Cell Metab. 26:809-811, 2017; Salzer et al. Cell 175:1575-1590.e22, 2018). To understand the effects of CGI status on age-associated increased transcriptional noise, a systematic meta-analysis of published scRNA-seq data generated from various human and mouse tissues/cells was performed (see, e.g., Bahar et al. Nature 441:1011-1014, 2006; Martinez-Jimenez et al. Science (80- ) 355:1433-1436, 2017; Enge et al. Cell 171:321-330.e14, 2017). The transcriptional noise of all genes was found to increase during aging both in human and mouse (FIG. 9). When the noise levels were separately monitored in CGI+ or CGI- genes alone, only CGI- genes, but not CGI+ genes, showed a dramatic increase in transcriptional noise during aging. These results indicated that dysregulation of CGI- genes is the driving factor of increased transcriptional noise during aging.

The association of CGI+ and CGI- genes with the Cellular Component (CC) sub-ontology from the Gene Ontology (GO) having distinct cellular localization patterns was tested. FIG. 10A data indicated that protein products of CGI+ and CGI- genes have significantly distinct localization patterns. CGI+ genes usually encoded intracellular proteins, while CGI- gene products were generally enriched with extracellular modulators (p = 1.7x10⁻⁹⁷), or plasma membrane-associated proteins (p = 2.7x10⁻⁹⁰). These data indicated that extracellular secretion is a characteristic of CGI- genes, rather than CGI+ genes.

Misexpressed CGI- genes encoding secretory factors were involved in various functions, including cytokines/chemokines, growth factors, proteases, and extracellular matrix components (FIG. 10B). It has been found that senescent cells are enriched in aged tissues, secrete various proteins with mixed functions causing systemic and local chronic inflammation. Misexpression of these secretory factors was clearly recapitulated upon nuclear lamina disruption, as well as in progeria and cellular senescence (FIG. 10D). These data suggested that nuclear architecture disruption and subsequent misexpression of CGI- genes may play an important role in the production of senescence-associated secretory phenotype (SASP) factors, as well as in age-associated chronic inflammation.

Specifically, mRNA-FISH (as in FIG. 10C) assays were used to test whether nuclear lamina disruption in the heart directly causes misexpression of CGI- genes encoding pro-inflammatory SASP factors. Kidney (affected DO) tissues from mice were used to test the genes that were recurrently found to be upregulated in a transcriptome analysis (FIGS. 10B, 10D), including tumor necrosis factor α (Tnf: up in DO, Lhr-KO/Hp1α-KD/progeria/senescence), interleukin 6 (I16: up in DO, Hp1α-KD/senescence), interleukin 10 (I110: up in DO, Lbr-KO/Hp1α-KD) and C-C motif chemokine ligand 5 (Ccl5: up in DO, Lmnb1-KD/Hp1α-KD/senescence). Four biological replicates from both sexes will be tested.

FIG. 10E shows the changes in the composition of the plasma proteome during aging, with the majority of CGI- gene products dramatically increased in abundance compared to CGI+ gene products. These data indicated that misexpression of CGI- genes significantly contributes to age-associated plasma proteome changes.

Since CGI- gene misexpression was commonly observed in various age-associated diseases (FIG. 7), nuclear architecture disruption and the resulting CGI- gene misexpression was suggested to directly cause organ dysfunction during aging.

### Example 8: Pharmacochemical Inhibition of Cgi- Gene Misexpression Delays Age-Associated Degenerative Changes

A massive-scale in silico drug repositioning analysis using published mRNA-seq data (generated from ~500 K conditions from ~24 K datasets) to scan for a condition that suppresses CGI-gene expression was performed. Inhibitors of BET (bromodomain and extra terminal domain) family proteins, such as JQ1, were found to selectively suppress CGI- gene expression.

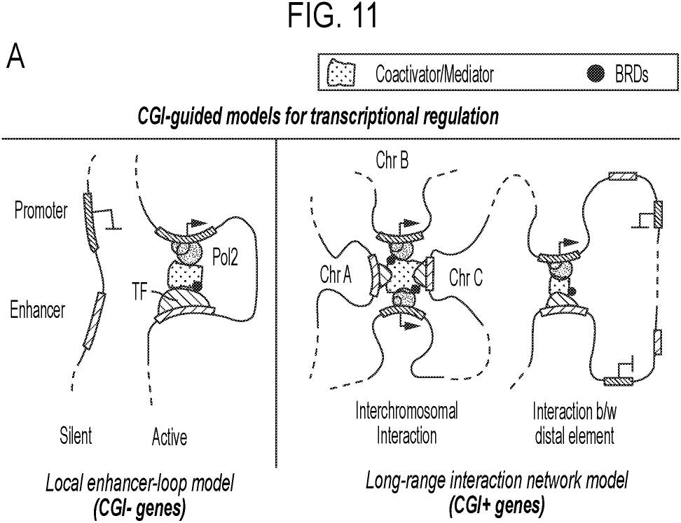

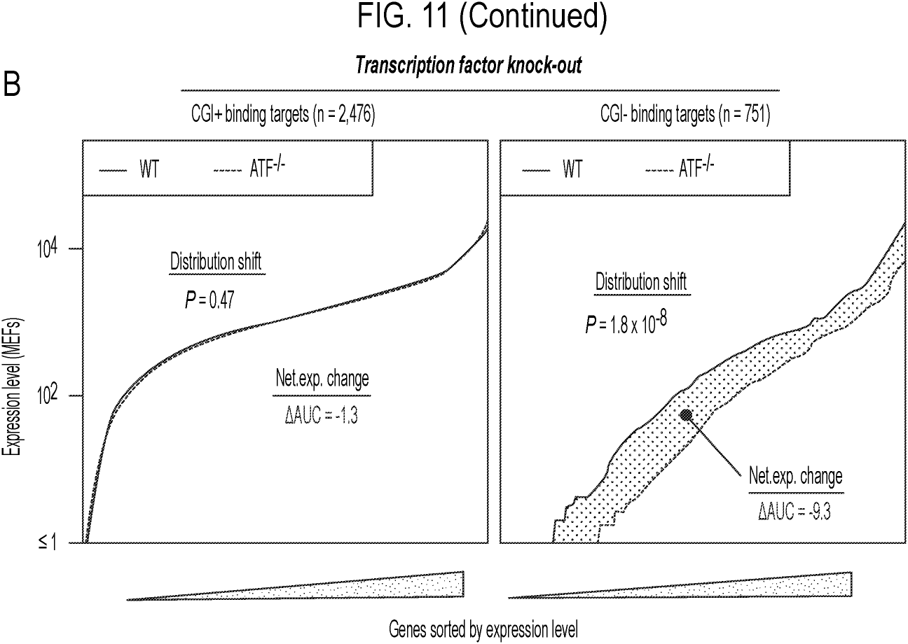

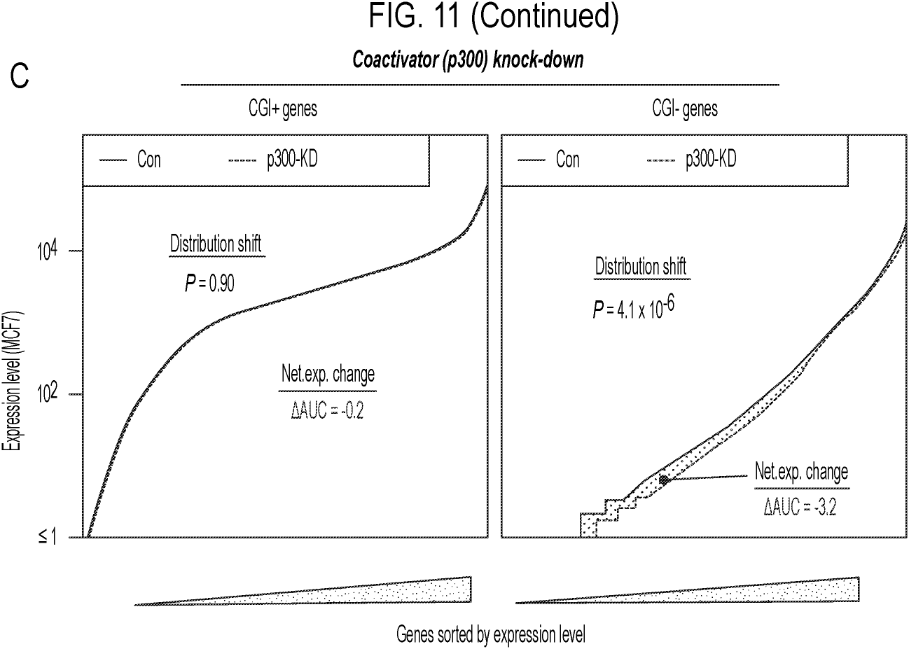

CGI- genes were found to be regulated by transcriptional regulation mechanisms distinct from those of CGI+ genes (FIG. 11A). CGI- genes were directly regulated through local enhancer-promoter interaction, while CGI+ genes were regulated by complex long-range chromatin interactions. As a result, CGI+ and CGI- genes exhibited very different responses to transcription factor (TF) perturbation (FIG. 11B). The depletion of TFs did not significantly affect the expression levels of CGI+ target genes. On the other hand, CGI- target genes showed dramatic downregulation, upon depletion of TFs. The depletion of the general component of local transcriptional activation, such as coactivator (p300), was shown to effectively suppress global CGI- gene expressions (FIG. 11C).

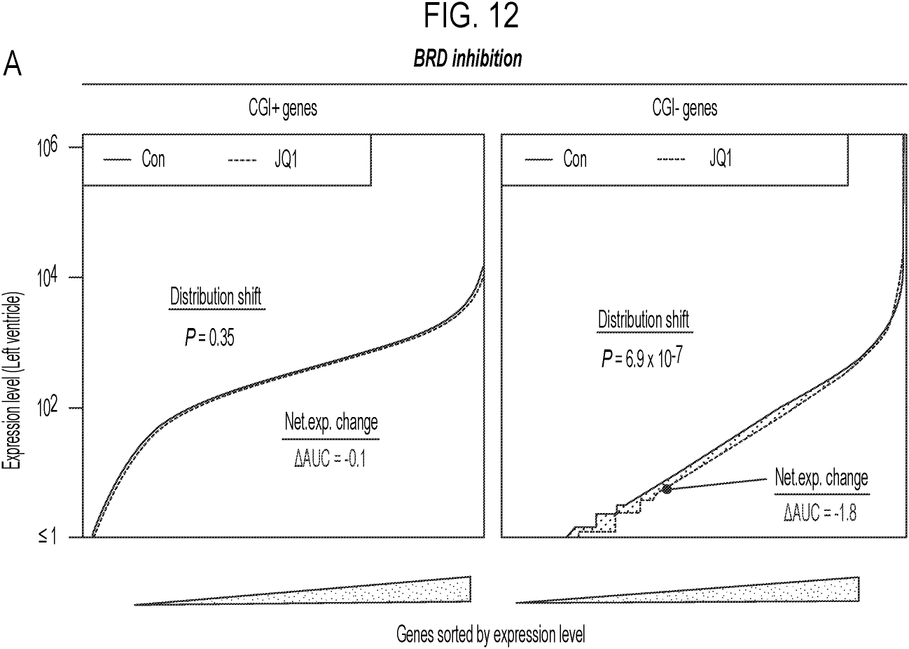

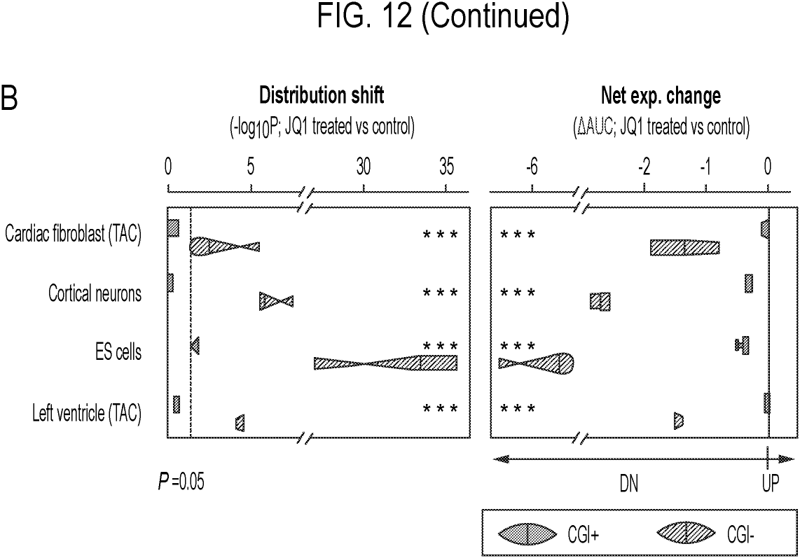

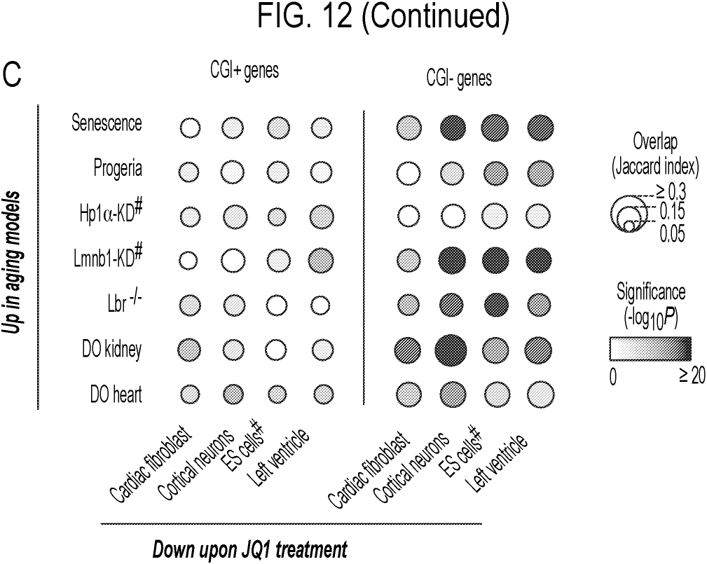

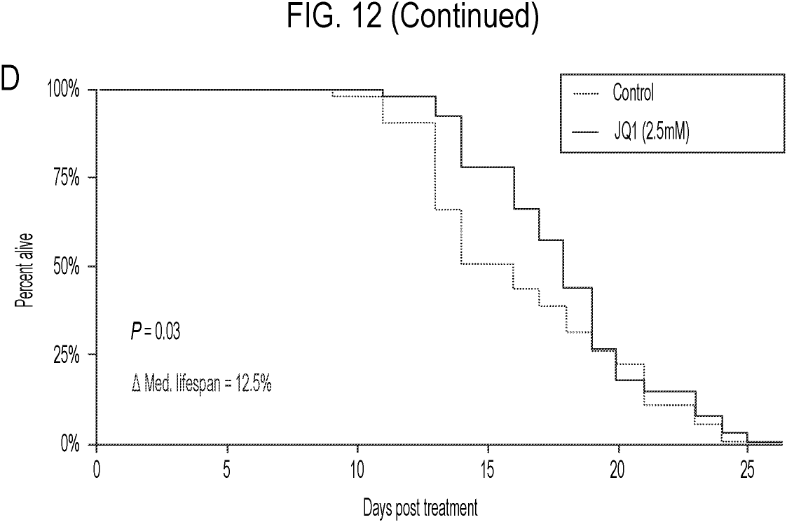

BET family proteins (e.g., BRD2/4) are also components of local enhancer mediated transcriptional activation (FIG. 11A). Treatment with a BET inhibitor, such as JQ1, resulted in global downregulation of CGI- gene expression, while CGI+ genes were not significantly affected (FIGS. 12A-12B). JQ1 and derivative BET inhibitors were found to be safe for humans and are currently undergoing clinical tests as anti-cancer drugs for various tumor types (Duan et al. Sci. Trans. Med, 9(390):eaah5084, 2017; Maksylewicz et al. Fron. Immunol. 10:933, 2019; Andrieu et al. 19:45-50, 2016). JQ1 has been shown to have anti-inflammatory effects (Ip, B. et al. Obesity 24(1):102-112, 2016; Mele et al. J. Exp. Med. 210(11):2181-2190, 2013), which aligned with the observation that CGI- gene misexpression may be associated with chronic inflammation during aging (FIG. 10).

FIG. 12C indicated that CGI- genes downregulated by JQ1 treatment largely overlapped with CGI- genes that were upregulated during aging or upon nuclear architecture disruption. This suggested that BET inhibitor treatment would effectively suppress age-associated CGI- gene misexpression. The effects of JQ1 on C. elegans lifespan was also tested. Although C. elegans do not have CGI elements in their genome, they do have heterochromatin-regulated genes, which are similar to mammalian CGI- genes. As shown in FIG. 12D, one-time treatment of JQ1 significantly extended the median lifespan of adult C. elegans. In addition, resveratrol, was shown to have pan-BET inhibitor activity (Dutra et al. Nutrients 9(11):1172, 2017, doi: 10.3390/nu9111172). Resveratrol was also screened to suppress CGI- gene expression. Treatment with BET inhibitors can suppress misexpression of CGI- genes during aging, and that this, in turn, will delay age-associated degenerative changes in mice.

Mice were treated by intraperiotoneal injections of 50 mg/kg/day of JQ1 as indicated in FIG. 13 demonstrating suppression of CGI- gene expression. All of the data were based on published RNA-seq data and compared to untreated controls for CGI+ and CGI- genes.

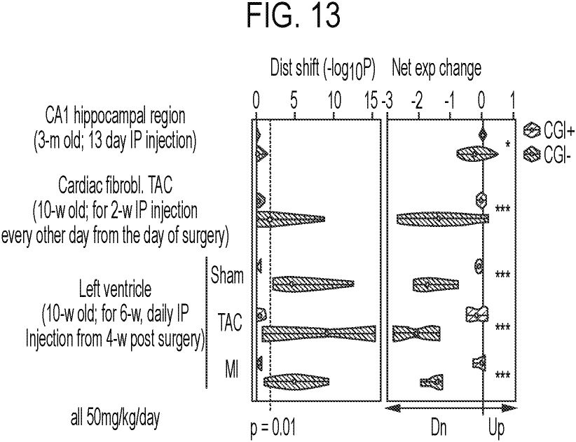

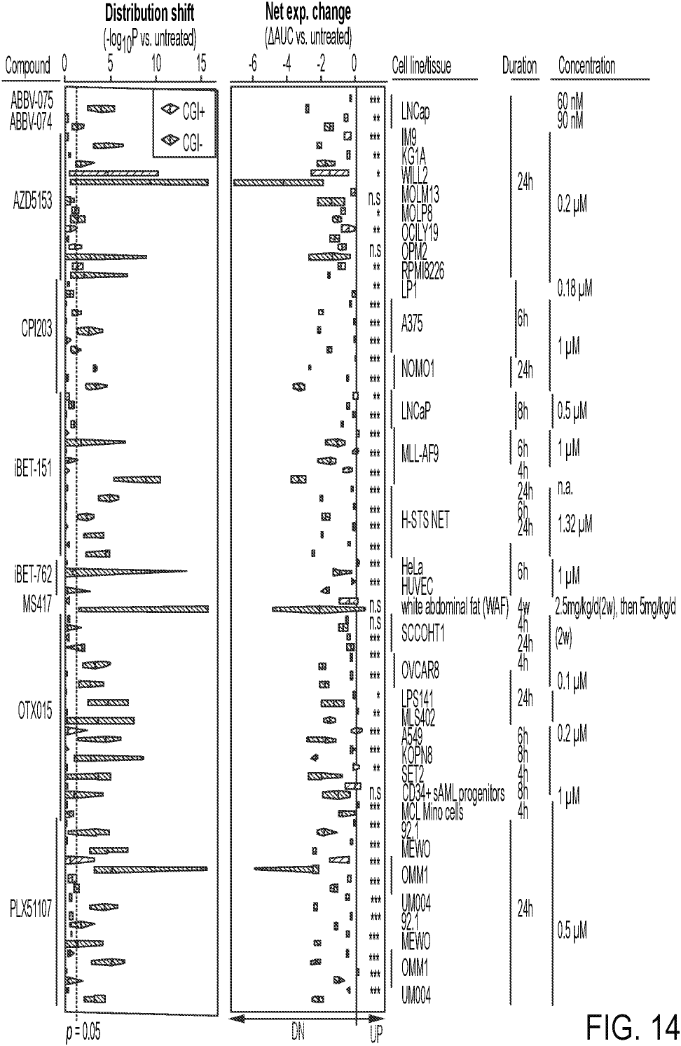

FIG. 14 demonstrated that CGI- gene expression was also suppressed by other BET inhibitors other than JQ1. Moreover, the cell line or tissue used in the BET inhibitor treatment, as well as treatment duration and concentration were also provided.

### Other Embodiments

As various changes can be made in the above-described subject matter without departing from the scope and spirit of the present disclosure, it is intended that all subject matter contained in the above description, or defined in the appended claims, be interpreted as descriptive and illustrative of the present disclosure. From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All documents cited or referenced herein and all documents cited or referenced in the herein cited documents, including all patents, publications, any manufacturer’s instructions, descriptions, product specifications, and product sheets for any products mentioned herein or in any document incorporated by reference herein, are herein incorporated by reference to the same extent as if each independent patent and publication was specifically and individually indicated to be incorporated by reference, and may be employed in the practice of the disclosure.

What is claimed is:

1. A method of treating a subject suffering from an age-related inflammatory disease in a subject, comprising administering a therapeutically effective amount of a Bromodomain and ExtraTerminal (BET) domain inhibitor to the subject in need thereof.

2. The method of claim 1, wherein the BET inhibitor is selected from tert-butyl (S)-2-(4-(4-chlorophenyl)-2,3,9-trimethyl-6H-thieno[3,2-f][1,2,4]triazolo[4,3-a][1,4]diazepin-6-yl)acetate (JQ1); (R)-7-(3,5-dimethylisoxazol-4-yl)-8-methoxy-1-(1-(pyridin-2-yl)ethyl)-1,3-dihydro-2H-imidazo[4,5-c]quinolin-2-one (I-BET 151); (S)-2-(6-(4-chlorophenyl)-8-methoxy-1-methyl-4H-benzo[f][1,2,4]triazolo [4,3-a][1,4]diazepin-4-yl)-N-ethylacetamide (I-BET 762); (S)-2-(6-(4-chlorophenyl)-8-methoxy-1-methyl-4H-benzo[f][1,2,4]triazolo[4,3-a][1,4]diazepin-4-yl)-N-ethylacetamide (OTX-015); (S)-2-(4-(4-chlorophenyl)-2,3,9-trimethyl-6H-thieno[3,2-f][1,2,4]triazolo[4,3-a][1,4]diazepin-6-yl)acetamide (CPI-203); (S)-2-(6-(4-chlorophenyl)-1-methyl-4H-benzo[c]isoxazolo[4,5-e]azepin-4-yl)acetamide (CPI-0610); 2-(4-(2-hydroxyethoxy)-3,5-dimethylphenyl)-5,7-dimethoxyquinazolin-4(3H)-one (RVX-208); (Z)-4-(2-(2-amino-5-methyl-4-oxocyclohexa-2,5-dien-1-ylidene)hydrazineyl)-N-(pyridin-2-yl)benzenesulfonamide (MS436); 2-methoxy-N-(3-methyl-2-oxo-1,2,3,4-tetrahydroquinazolin-6-yl)benzenesulfonamide (PFI-1); N-ethyl-4-(2-(4-fluoro-2,6-dimethylphenoxy)-5-(2-hydroxypropan-2-yl)phenyl)-6-methyl-7-oxo-6,7-dihydro-1H-pyrrolo[2,3-c]pyridine-2-carboxamide (ABBV-744); 2-Morpholin-4-yl-8-phenylchromen-4-one (LY294002); (R)-4-(2-(4-(1-(3-methoxy-[1,2,4]triazolo[4,3-b]pyridazin-6-yl)piperidin-4-yl)phenoxy) ethyl)-1,3-dimethylpiperazin-2-one (AZD 5153); N,N′-(3,6,9,12,15,18,21-heptaoxatricosane-1,23-diyl)bis(2-((S)-4-(4-chlorophenyl)-2,3,9-trimethyl-6H-thieno[3,2-f][1,2,4]triazolo[4,3-a][1,4] diazepin-6-yl) acetamide) (MT-1); (S)-N,N′-(decane-1,10-diyl)bis(2-((6S)-4-(4-chlorophenyl)-2,3,9-trimethyl-6H-thieno[3,2-f] [1,2,4] triazolo[4,3-a][1,4]diazepin-6-yl)acetamide) (MS645); 2-(4-(2-(isopropylamino)ethoxy)-3,5-dimethylphenyl)-5,7-dimethoxyquinazolin-4(3H)-one (RVX2135); (S)-7,8-dimethoxy-N,4-dimethyl-1-(4-(4-methylpiperazin-1-yl)phenyl)-4,5-dihydro-3H-benzo[d] [1,2]diazepine-3-carboxamide (BAY1238097); (S)-6-(3,5-dimethylisoxazol-4-yl)-3-(pyridin-2-yl)-3,4-dihydro-5-oxa-1,2a-diazaacenaphthylen-2(1H)-one (INCB054329); 6-(3-hydroxypropyl)-2-(1,3,6-trimethyl-2-oxo-2,3-dihydro-1H-benzo[d]imidazol-5-yl)-1H-benzo[de]isoquinoline-1,3(2H)-dione (BAY-299); (S)-2-(3-(1,4-dimethyl-1H-1,2,3-triazol-5-yl)-5-(phenyl(tetrahydro-2H-pyran-4-yl)methyl)-5H-pyrido[3,2-b]indol-7-yl)propan-2-ol (BMS-986158); N-(4-(2,4-difluorophenoxy)-3-(6-methyl-7-oxo-6,7-dihydro-1H-pyrrolo[2,3-c]pyridin-4-yl)phenyl)ethanesulfonamide (ABBV-075); (2-cyclopropyl-5-(3,5-dimethyl-3H-1l3-isoxazol-4-yl)-1H-benzo[d]imidazol-7-yl)di(pyridin-2-yl) methanol (GS-5829, Alobresib); (S)-4-(6-(3,5-dimethylisoxazol-4-yl)-1-(1-(pyridin-2-yl)ethyl)-1H-pyrrolo[3,2-b]pyridin-3-yl)benzoic acid (PLX51107); methyl (S)-2-(4-(4-chlorophenyl)-2,3,9-trimethyl-6H-thieno[3,2-f][1,2,4]triazolo[4,3-a][1,4]diazepin-6-yl)acetate (MS417); TEN-010; ZEN003694; GSK2820151; FT-1101; olinone; 2-((S)-4-(4-chlorophenyl)-2,3,9-trimethyl-6H-thieno[3,2-f][1,2,4]triazolo [4,3-a][1,4]diazepin-6-yl)-N-(4-(2-(2-(2-(2-((2-(2,6-dioxopiperidin-3-yl)-1,3-dioxoisoindolin-4-yl) amino)ethoxy)ethoxy)ethoxy) ethoxy)phenyl)acetamide (ARV-825); 2-[[[4-(1,2-Dihydro-2-methyl-1-oxo-2,7-naphthyridin-4-yl)-2-6-dimethoxyphenyl]methyl]methylamino]-N-[2-[2-[2-[[2-(2,6-dioxo-3-piperidinyl)-2,3-dihydro-1,3-dioxo-1H-isoindol-4-yl]amino]ethoxy]ethoxy]ethyl] acetamide dihydrochloride (dBRD9); or pharmaceutically acceptable enantiomers, diastereomers, racemates, and salts thereof.

3. The method of claim 1 1-2, wherein the BET inhibitor is tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof.

4. A method of treating a subject suffering from an age-related inflammatory disease in a subject, comprising administering a therapeutically effective amount of tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof, to the subject in need thereof.

5. The method of claim 11, 1–4 wherein the method delays aging.

6. The method of claim 11, 1–4 wherein the method delays the onset of the age-related inflammatory disease.

7. The method of claim 1 1, wherein the method extends lifespan of the subject.

8. The method of claim 7, wherein lifespan increases by 1% - 50%.

9. The method of claim 1, 1 wherein the age-related inflammatory disease is selected from Alzheimer’s disease, Parkinson’s, chronic inflammation, Rheumatoid arthritis, maculopathy atherosclerosis, diabetes, stroke, myocardial infarction, heart failure, hypertension, osteoarthritis, osteoporosis, sarcopenia, loss of bone marrow, idiopathic pulmonary fibrosis, degraded immune function, age-related macular degeneration, abnormal proliferative diseases, and disorders associated with a decrease in hormones or reduced energy production.

10. The method of claim 1 1–9, wherein the subject is a mammal.

11. The method of claim 11 1–10, wherein the subject is human.

12. A method of inhibiting Cytosine-phosphate-Guanine negative (CGI-) gene misexpression in a cell, comprising administering an effective amount of a BET inhibitor to the cell.

13. The method of claim 12, wherein the BET inhibitor is selected from tert-butyl (S)-2-(4-(4-chlorophenyl)-2,3,9-trimethyl-6H-thieno[3,2-f][1,2,4]triazolo[4,3-a][1,4]diazepin-6-yl)acetate; (R)-7-(3,5-dimethylisoxazol-4-yl)-8-methoxy-1-(1-(pyridin-2-yl)ethyl)-1,3-dihydro-2H-imidazo[4,5-c] quinolin-2-one; (S)-2-(6-(4-chlorophenyl)-8-methoxy-1-methyl-4H-benzo[f][1,2,4]triazolo [4,3-a] [1,4]diazepin-4-yl)-N-ethylacetamide; (S)-2-(6-(4-chlorophenyl)-8-methoxy-1-methyl-4H-benzo[f] [1,2,4]triazolo[4,3-a][1,4]diazepin-4-yl)-N-ethylacetamide; (S)-2-(4-(4-chlorophenyl)-2,3,9-trimethyl-6H-thieno[3,2-f][1,2,4]triazolo[4,3-a][1,4]diazepin-6-yl)acetamide; (S)-2-(6-(4-chlorophenyl)-1-methyl-4H-benzo[c]isoxazolo [4,5-e]azepin-4-yl)acetamide; 2-(4-(2-hydroxyethoxy)-3,5-dimethylphenyl)-5,7-dimethoxyquinazolin-4(3H)-one; (Z)-4-(2-(2-amino-5-methyl-4-oxocyclohexa-2,5-dien-1-ylidene)hydrazineyl)-N-(pyridin-2-yl)benzenesulfonamide; 2-methoxy-N-(3-methyl-2-oxo-1,2,3,4-tetrahydroquinazolin-6-yl)benzenesulfonamide; N-ethyl-4-(2-(4-fluoro-2,6-dimethylphenoxy) -5-(2-hydroxypropan-2-yl)phenyl)-6-methyl-7-oxo-6,7-dihydro-1H-pyrrolo[2,3-c]pyridine-2-carboxamide; 2-Morpholin-4-yl-8-phenylchromen-4-one; (R)-4-(2-(4-(1-(3-methoxy-[1,2,4] triazolo[4,3-b]pyridazin-6-yl)piperidin-4-yl)phenoxy) ethyl)-1,3-dimethylpiperazin-2-one; N,N′-(3,6,9,12,15,18,21-heptaoxatricosane-1,23-diyl)bis(2-((S)-4-(4-chlorophenyl)-2,3,9-trimethyl-6H-thieno[3,2-f][1,2,4] triazolo[4,3-a][1,4] diazepin-6-yl) acetamide); (S)-N,N′-(decane-1,10-diyl)bis(2-((6S)-4-(4-chlorophenyl)-2,3,9-trimethyl-6H-thieno[3,2-f][1,2,4]triazolo[4,3-a][1,4] diazepin-6-yl)acetamide); 2-(4-(2-(isopropylamino)ethoxy)-3,5-dimethylphenyl)-5,7-dimethoxyquinazolin-4(3H)-one; (S)-7,8-dimethoxy-N,4-dimethyl-1-(4-(4-methylpiperazin-1-yl)phenyl)-4,5-dihydro-3H-benzo[d] [1,2]diazepine-3-carboxamide; (S)-6-(3,5-dimethylisoxazol-4-yl)-3-(pyridin-2-yl)-3,4-dihydro-5-oxa-1,2a-diazaacenaphthylen-2(1H)-one; 6-(3-hydroxypropyl)-2-(1,3,6-trimethyl-2-oxo-2,3-dihydro-1H-benzo [d]imidazol-5-yl)-1H-benzo[de]isoquinoline-1,3(2H)-dione; (S)-2-(3-(1,4-dimethyl-1H-1,2,3-triazol-5-yl)-5-(phenyl(tetrahydro-2H-pyran-4-yl)methyl)-5H-pyrido[3,2-b]indol-7-yl)propan-2-ol; N-(4-(2,4-difluorophenoxy)-3-(6-methyl-7-oxo-6,7-dihydro-1H-pyrrolo[2,3-c]pyridin-4-yl)phenyl)ethanesulfonamide; (2-cyclopropyl-5-(3,5-dimethyl-3H-1l3-isoxazol-4-yl)-1H-benzo[d]imidazol-7-yl)di (pyridin-2-yl) methanol; (S)-4-(6-(3,5-dimethylisoxazol-4-yl)-1-(1-(pyridin-2-yl)ethyl)-1H-pyrrolo [3,2-b]pyridin-3-yl)benzoic acid; methyl (S)-2-(4-(4-chlorophenyl)-2,3,9-trimethyl-6H-thieno[3,2-f][1,2,4]triazolo[4,3-a][1,4]diazepin-6-yl)acetate; ZEN003694; GSK2820151; FT-1101; TEN-010; olinone; 2-((S)-4-(4-chlorophenyl)-2,3,9-trimethyl-6H-thieno[3,2-f][1,2,4]triazolo [4,3-a][1,4]diazepin-6-yl)-N-(4-(2-(2-(2-(2-((2-(2,6-dioxopiperidin-3-yl)-1,3-dioxoisoindolin-4-yl) amino) ethoxy)ethoxy)ethoxy) ethoxy)phenyl)acetamide; 2-[[[4-(1,2-Dihydro-2-methyl-1-oxo-2,7-naph thyridin-4-yl)-2-6-dimethoxyphenyl]methyl]methylamino]-N-[2-[2-[2-[[2-(2,6-dioxo-3-piperidinyl)-2,3-dihydro-1,3-dioxo-1H-isoindol-4-yl]amino]ethoxy]ethoxy]ethyl] acetamide dihydrochloride or pharmaceutically acceptable enantiomers, diastereomers, racemates, and salts thereof.

14. The method of claim 12 12–13, wherein the BET inhibitor is tert-butyl (S)-2-(4-(4-chlorophenyl)-2,3,9-trimethyl-6H-thieno[3,2-f][1,2,4]triazolo[4,3-a][1,4]diazepin-6-yl)acetate, or pharmaceutically acceptable enantiomers, diastereomers, racemates, and salts thereof.

15. The method of claim 12 12–14, wherein the cell is a senescent cell.

16. The method of claim 12 12–15, wherein the cell is present in an age-related inflammatory disease.

17. The method of claim 16, wherein the age-related inflammatory disease is selected from Alzheimer’s disease, Parkinson’s, chronic inflammation, Rheumatoid arthritis, maculopathy atherosclerosis, diabetes, stroke, myocardial infarction, heart failure, hypertension, osteoarthritis, osteoporosis, sarcopenia, loss of bone marrow, idiopathic pulmonary fibrosis, degraded immune function, age-related macular degeneration, abnormal proliferative diseases, and disorders associated with a decrease in hormones or reduced energy production.

18. The method of claim 12 12, wherein the method inhibits nuclear lamina and/or heterochromatin disruption.

19. The method of claim 12 12, wherein the method inhibits Cytosine-phosphate-Guanine negative (CGI-) misexpression associated organ failure.

20. The method of claim 12 12, wherein the method inhibits age-associated degenerative changes.
